(19) United States
(12) Patent Application Publication
(10) Pub. No.: US 2026/0366003 A1
(43) Pub. Date: Nov. 19, 2026

(54) POLYPEPTIDE HAVING LYSOZYME ACTIVITY AND POLYNUCLEOTIDES ENCODING SAME

(71) Applicant: NOVOZYMES A/S, Bagsvaerd (DK)

(72) Inventors: Rolf Thomas LENHARD, Lyngby (DK); Anne Veller Friis STISSING, Vaerloese (DK); Lars Kobberoee SKOV, Ballerup (DK)

(73) Assignee: NOVOZYMES A/S, Bagsvaerd (DK)

(21) Appl. No.: 19/133,592

(22) PCT Filed: Dec. 7, 2023
(86) PCT No.: PCT/EP2023/084738

§ 371 Date: May 28, 2025

(30) Foreign Application Priority Data
Dec. 8, 2022 (EP) 22213122309.3

(51) Int. Cl. C12N 9/36; A23K 20/147; A23K 20/189; A23K 20/20

(52) U.S. Cl. CPC C12N 9/2462; A23K 20/147; A23K 20/189; A23K 20/20; C12Y 302/01017

(57) ABSTRACT

The present invention relates to a thermostable polypeptide having lysozyme activity and polynucleotides encoding the polypeptides. The invention also relates to nucleic acid constructs, vectors, and host cells comprising the polynucleotides as well as methods of producing and using the polypeptides.

### REFERENCE TO A SEQUENCE LISTING

This application contains a Sequence Listing in computer readable form, which is incorporated herein by reference.

### FIELD OF THE INVENTION

The present invention relates to polypeptides having lysozyme activity, and polynucleotides encoding the polypeptides. The invention also relates to nucleic acid constructs, vectors, and host cells comprising the polynucleotides as well as methods of producing and using the polypeptides.

### BACKGROUND OF THE INVENTION

Lysozyme is a O-glycosyl hydrolase produced as a defensive mechanism against bacteria by many organisms. The enzyme causes the hydrolysis of bacterial cell walls by cleaving the glycosidic bonds of peptidoglycan; an important structural molecule in bacteria. After having their cell walls weakened by lysozyme action, bacterial cells lyse resulting from osmotic pressure. Lysozyme occurs in many organisms such as viruses, plants, insects, birds, reptiles and mammals. In mammals, Lysozyme has been isolated from nasal secretions, saliva, tears, intestines, urine and milk. The enzyme cleaves the glycosidic bond between carbon number 1 of N-acetylmuramic acid and carbon number 4 of N-acetyl-D-glucosamine. In vivo, these two carbohydrates are polymerized to form the cell wall polysaccharide.

Lysozyme is typically produced as a defensive mechanism against bacteria by many organisms as viruses, plants, insects, birds, reptiles and mammals. The enzyme causes the hydrolysis of bacterial cell walls by cleaving the glycosidic bonds of peptidoglycan; an important structural molecule in bacteria. After having their cell walls weakened by lysozyme action, bacterial cells lyse resulting from osmotic pressure. There is an increasing interest in the potential of lysozyme enzymes as antimicrobial agents. For example, lysozyme activity has been shown against pathogens such as Streptococcus pneumoniae, Bacillus anthracis, Enterococcus faecium, Bacillus stearothermophilus, Clostridium botulinum, Clostridium butyricum, Clostridium perfringens, Clostridium sporogenes, Clostridium tyrobutyricum, and Listeria monocytogenes.

Lysozyme has been classified into five different glycoside hydrolase (GH) families (CAZy, www.cazy.org): hen egg-white lysozyme (GH22), goose egg-white lysozyme (GH23), bacteriophage T4 lysozyme (GH24), Sphingomonas flagellar protein (GH73) and Chalaropsis lysozymes (GH25). Lysozymes from the families GH23 and GH24 are primarily known from bacteriophages and have not been identified in fungi. The lysozyme family GH25 has been found to be structurally unrelated to the other lysozyme families.

Use of lysozyme has been suggested in animal feed (see for example WO 00/21381 and WO 04/026334), in cheese production (see for example WO 05/080559), food preservation (Hughey and Johnson (1987) Appl Environ Microbiol 53:2165), detergents (see for example U.S. Pat. No. 5,041,236 and EP 0425016), in oral care (see for example U.S. Pat. No. 4,355,022, WO 04/017988 and WO 08/124764), cosmetology and dermatology, contraception, urology, and gynaecology (see for example WO 08/124764).

A GH25 lysozyme has been reported from Chalaropsis (Felsch J W, Ingagami T, and Hash J H. (1975) The N,O-Diacetylmuramidase of Chalaropsis species; V The complete amino acid sequence. JBC. 250:10 pp 3713-3720).

Hen egg white lysozyme which is the primary product available on the commercial market, does not cleave N,6-O-diacetylmuramidase in e.g. Streptococcus aureus cell walls and is thus unable to lyse this important human pathogen among others.

It has been observed that different lysozymes have different specificities towards different microorganisms. It is therefore desirable to have several lysozymes available in order to be able to select suitable enzymes for each particular application. New polypeptides having lysozyme activity is therefore desired. For example, many applications in which lysozyme is used, undergo high temperature process, and a therefore there is a need for thermostable lysozyme.

### SUMMARY OF THE INVENTION

The present invention relates to polypeptides having lysozyme activity and nucleotide sequences encoding the polypeptides. In one aspect, the polypeptide comprising a substitution at one or more positions corresponding to positions 1, 2, 6, 9, 10, 13, 14, 20, 36, 37, 40, 41, 63, 65, 101, 103, 118, 122, 125, 126, 129, 131, 143, 153, 155, 166, 174, 175, 176, 188 and 202 of SEQ ID NO: 1, wherein the polypeptide has at least 60%, e.g., at least 65%, at least 70%, at least 75%, at least 80%, at least 85%, at least 90%, at least 95%, at least 96%, at least 97%, at least 98%, or at least 99% sequence identity, but less than 100% sequence identity, to the polypeptide of SEQ ID NO: 1 or amino acids 20 to 227 of SEQ ID NO: 2. The polypeptide has improved thermostable properties compared to the polypeptide having SEQ ID NO: 1 or amino acids 20 to 227 of SEQ ID NO: 2.

In another aspect, the present invention relates to polynucleotides encoding the polypeptide; nucleic acid constructs, vectors, and host cells comprising the polynucleotides; and methods of producing the polypeptide.

The invention is further directed to a composition comprising the polypeptide as defined herein.

An interesting aspect of the invention is directed to an animal feed additive comprising the polypeptide of the invention; and at least one fat soluble vitamin, and/or at least one water soluble vitamin, and/or at least one trace mineral.

In yet another aspect, the present invention also relates to use of the polypeptide of the present invention in animal feed.

An aspect of the invention is directed to the use of the polypeptide of the invention:
i. in animal feed;
ii. in animal feed additives;
iii. in the preparation of a composition for use in animal feed; and/or
iv. for improving the intestinal health in an animal.

### Overview of Sequence Listing

SEQ ID NO:1 is amino acid sequence of lysozyme isolated from Sodiomyces alcalophilus.
SEQ ID NO:2 is mature amino acid sequence of the lysozyme isolated from Sodiomyces alcalophilus.
SEQ ID NO:3 is nucleic acid sequence of the lysozyme isolated from Sodiomyces alcalophilus.

### BRIEF DESCRIPTION OF THE FIGURES

FIG. 1 shows a graph that demonstrates residual activity of the polypeptide of the invention and wildtype in steam box conditions.

### DETAILED DESCRIPTION OF THE INVENTION

The present invention relates to polypeptides having lysozyme activity, and polynucleotides encoding the polypeptides. The polypeptide having lysozyme activity can be defined as a variant of SEQ ID NO:1 or SEQ ID NO:2, and comprises a substitution at one or more positions corresponding to position 1, 2, 6, 9, 10, 13, 14, 20, 36, 37, 40, 41, 63, 65, 101, 103, 118, 122, 125, 126, 129 131, 143, 153, 155, 166 174, 175, 176, 188 and 202 of SEQ ID NO: 1, wherein the polypeptide has at least 60% sequence identity to SEQ ID NO:1, or amino acids 20 to 227 of SEQ ID NO: 2.

### DEFINITIONS

In accordance with this detailed description, the following definitions apply. Note that the singular forms “a,” “an,” and “the” include plural references unless the context clearly dictates otherwise.

Unless defined otherwise or clearly indicated by context, all technical and scientific terms used herein have the same meaning as commonly understood by one of ordinary skill in the art to which this invention belongs.

Lysozyme: The term “lysozyme” means a peptidoglycan N-acetylmuramoylhydrolase (EC 3.2.1.17) that catalyzes the hydrolysis of 1,4-beta-linkages between N-acetylmuramic acid and N-acetyl-D-glucosamine residues in a peptidoglycan and between N-acetyl-D-glucosamine residues in chitodextrins.

Lysozyme activity: The term “lysozyme activity” is defined herein as a peptidoglycan N-acetylmuramoylhydrolase activity (EC 3.2.1.17) that catalyzes the hydrolysis of 1,4-beta-linkages between N-acetylmuramic acid and N-acetyl-D-glucosamine residues in a peptidoglycan and between N-acetyl-D-glucosamine residues in chitodextrins.

cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Coding sequence: The term “coding sequence” means a polynucleotide, which directly specifies the amino acid sequence of a variant. The boundaries of the coding sequence are generally determined by an open reading frame, which begins with a start codon such as ATG, GTG or TTG and ends with a stop codon such as TAA, TAG, or TGA. The coding sequence may be a genomic DNA, cDNA, synthetic DNA, or a combination thereof.

Control sequences: The term “control sequences” means nucleic acid sequences involved in regulation of expression of a polynucleotide in a specific organism or in vitro. Each control sequence may be native (i.e., from the same gene) or heterologous (i.e., from a different gene) to the polynucleotide encoding the variant, and native or heterologous to each other. Such control sequences include, but are not limited to leader, polyadenylation, prepropeptide, propeptide, signal peptide, promoter, terminator, enhancer, and transcription or translation initiator and terminator sequences. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a variant.

Expression: The term “expression” includes any step involved in the production of a variant including, but not limited to, transcription, post-transcriptional modification, translation, post-translational modification, and secretion.

Expression vector: An “expression vector” refers to a linear or circular DNA construct comprising a DNA sequence encoding a variant, which coding sequence is operably linked to a suitable control sequence capable of effecting expression of the DNA in a suitable host. Such control sequences may include a promoter to effect transcription, an optional operator sequence to control transcription, a sequence encoding suitable ribosome binding sites on the mRNA, enhancers and sequences which control termination of transcription and translation.

Extension: The term “extension” means an addition of one or more amino acids to the amino and/or carboxyl terminus of a variant, wherein the “extended” variant has lysozyme activity.

Fragment: The term “fragment” means a variant having one or more amino acids absent from the amino and/or carboxyl terminus of the variant; wherein the fragment has lysozyme activity.

Fusion polypeptide: The term “fusion polypeptide” is a polypeptide in which one polypeptide is fused at the N-terminus and/or the C-terminus of a variant of the present invention. A fusion polypeptide is produced by fusing a polynucleotide encoding another polypeptide to a polynucleotide of the present invention, or by fusing two or more polynucleotides of the present invention together. Techniques for producing fusion polypeptides are known in the art and include ligating the coding sequences encoding the polypeptides so that they are in frame and that expression of the fusion polypeptide is under control of the same promoter(s) and terminator. Fusion polypeptides may also be constructed using intein technology in which fusion polypeptides are created post-translationally (Cooper et al., 1993, EMBO J. 12:2575-2583; Dawson et al., 1994, Science 266:776-779). A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3:568-576; Svetina et al., 2000, J. Biotechnol. 76:245-251; Rasmussen-Wilson et al., 1997, Appl. Environ. Microbiol. 63:3488-3493; Ward et al., 1995, Biotechnology 13:498-503; and Contreras et al., 1991, Biotechnology 9:378-381; Eaton et al., 1986, Biochemistry 25:505-512; Collins-Racie et al., 1995, Biotechnology 13:982-987; Carter et al., 1989, Proteins: Structure, Function, and Genetics 6:240-248; and Stevens, 2003, Drug Discovery World 4:35-48.

Heterologous: The term “heterologous” means, with respect to a host cell, that a polypeptide or nucleic acid does not naturally occur in the host cell. The term “heterologous” means, with respect to a polypeptide or nucleic acid, that a control sequence, e.g., promoter, of a polypeptide or nucleic acid is not naturally associated with the polypeptide or nucleic acid, i.e., the control sequence is from a gene other than the gene encoding the mature polypeptide.

Host Strain or Host Cell: A “host strain” or “host cell” is an organism into which an expression vector, phage, virus, or other DNA construct, including a polynucleotide encoding a variant has been introduced. Exemplary host strains are microorganism cells (e.g., bacteria, filamentous fungi, and yeast) capable of expressing the polypeptide of interest and/or fermenting saccharides. The term “host cell” includes protoplasts created from cells.

Improved thermostability: The term “improved thermostability” is defined herein as a variant enzyme displaying retention of enzymatic activity after a period of incubation at elevated temperature relative to the parent enzyme. Such a variant may or may not display an altered thermal activity profile relative to the parent. For example, the variant may not be active at the elevated temperatures but is able to maintain its three-dimensional structure and then regain activity once it is returned to lower temperatures. Alternatively, the variant may have an improved ability to refold following incubation at elevated temperature relative to the parent enzyme.

In one aspect, the thermostability of a lysozyme variant is improved such that the variant can survive high temperatures e.g. temperatures from 45 °C to 110 °C, preferably from 50 °C to 100 °C, more preferably from 60 °C to 90 °C, even more preferably from 70 °C to 80 °C. Preferably, the variant lysozyme maintains at least 40%, preferably at least 50%, 60%, 70% or 80%, more preferably at least 90%, even more preferably at least 95% residual activity after incubation at a given high temperature for 1 hour when compared to the variant which has been maintained at room temperature for the same time. Preferably, the residual activity of the variant lysozyme is at least 1.5-fold, preferably at least 2-fold, more preferably at least 5-fold, most preferably at least 7-fold, and even most preferably at least 20-fold higher than the residual activity of the parent lysozyme which has been treated under the same conditions. Preferably the activity is tested using the lysozyme activity assay described in the “Examples” section, with the deviation of the temperature to the desired increased temperature.

Introduced: The term “introduced” in the context of inserting a nucleic acid sequence into a cell, means “transfection”, “transformation” or “transduction,” as known in the art.

Isolated: The term “isolated” means a variant, nucleic acid, cell, or other specified material or component that is separated from at least one other material or component, including but not limited to, other proteins, nucleic acids, cells, etc. An isolated polypeptide, nucleic acid, cell or other material is thus in a form that does not occur in nature. An isolated polypeptide includes, but is not limited to, a culture broth containing the secreted variant expressed in a host cell.

Mature polypeptide: The term “mature polypeptide” means a polypeptide in its mature form following N-terminal processing and/or C-terminal processing (e.g., removal of signal peptide).

Mature polypeptide coding sequence: The term “mature polypeptide coding sequence” means a polynucleotide that encodes a mature polypeptide having lysozyme activity.

Mutant: The term “mutant” means a polynucleotide encoding a variant.

Native: The term “native” means a nucleic acid or polypeptide naturally occurring in a host cell.

Nucleic acid: The term “nucleic acid” encompasses DNA, RNA, heteroduplexes, and synthetic molecules capable of encoding a variant. Nucleic acids may be single stranded or double stranded and may be chemical modifications. The terms “nucleic acid” and “polynucleotide” are used interchangeably. Because the genetic code is degenerate, more than one codon may be used to encode a particular amino acid, and the present compositions and methods encompass nucleotide sequences that encode a particular amino acid sequence. Unless otherwise indicated, nucleic acid sequences are presented in 5′-to-3′ orientation.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature, or which is synthetic, and which comprises one or more control sequences operably linked to the nucleic acid sequence.

Operably linked: The term “operably linked” means that specified components are in a relationship (including but not limited to juxtaposition) permitting them to function in an intended manner. For example, a regulatory sequence is operably linked to a coding sequence such that expression of the coding sequence is under control of the regulatory sequence.

Parent or parent lysozyme: The term “parent” or “parent lysozyme” means a lysozyme to which an alteration is made to produce the enzyme variants of the present invention.

Purified: The term “purified” means a nucleic acid, variant or cell that is substantially free from other components as determined by analytical techniques well known in the art (e.g., a purified variant or nucleic acid may form a discrete band in an electrophoretic gel, chromatographic eluate, and/or a media subjected to density gradient centrifugation). A purified nucleic acid or variant is at least about 50% pure, usually at least about 60%, about 65%, about 70%, about 75%, about 80%, about 85%, about 90%, about 91%, about 92%, about 93%, about 94%, about 95%, about 96%, about 97%, about 98%, about 99%, about 99.5%, about 99.6%, about 99.7%, about 99.8% or more pure (e.g., percent by weight or on a molar basis). In a related sense, a composition is enriched for a molecule when there is a substantial increase in the concentration of the molecule after application of a purification or enrichment technique. The term “enriched” refers to a compound, variant, cell, nucleic acid, amino acid, or other specified material or component that is present in a composition at a relative or absolute concentration that is higher than a starting composition.

In one aspect, the term “purified” as used herein refers to the variant or cell being essentially free from components (especially insoluble components) from the production organism. In other aspects, the term “purified” refers to the variant being essentially free of insoluble components (especially insoluble components) from the native organism from which it is obtained. In one aspect, the variant is separated from some of the soluble components of the organism and culture medium from which it is recovered. The variant may be purified (i.e., separated) by one or more of the unit operations filtration, precipitation, or chromatography.

Accordingly, the variant may be purified such that only minor amounts of other proteins, in particular, other polypeptides, are present. The term “purified” as used herein may refer to removal of other components, particularly other proteins and most particularly other enzymes present in the cell of origin of the polypeptide. The variant may be “substantially pure”, i.e., free from other components from the organism in which it is produced, e.g., a host organism for recombinantly produced variant. In one aspect, the polypeptide is at least 40% pure by weight of the total polypeptide material present in the preparation. In one aspect, the polypeptide is at least 50%, 60%, 70%, 80% or 90% pure by weight of the total polypeptide material present in the preparation. As used herein, a “substantially pure polypeptide” may denote a polypeptide preparation that contains at most 10%, preferably at most 8%, more preferably at most 6%, more preferably at most 5%, more preferably at most 4%, more preferably at most 3%, even more preferably at most 2%, most preferably at most 1%, and even most preferably at most 0.5% by weight of other polypeptide material with which the polypeptide is natively or recombinantly associated.

It is, therefore, preferred that the substantially pure variant is at least 92% pure, preferably at least 94% pure, more preferably at least 95% pure, more preferably at least 96% pure, more preferably at least 97% pure, more preferably at least 98% pure, even more preferably at least 99% pure, most preferably at least 99.5% pure by weight of the total polypeptide material present in the preparation. The variant of the present invention is preferably in a substantially pure form (i.e., the preparation is essentially free of other polypeptide material with which it is natively or recombinantly associated). This can be accomplished, for example by preparing the variant by well-known recombinant methods or by classical purification methods.

Recombinant: The term “recombinant” is used in its conventional meaning to refer to the manipulation, e.g., cutting and rejoining, of nucleic acid sequences to form constellations different from those found in nature. The term recombinant refers to a cell, nucleic acid, variant or vector that has been modified from its native state. Thus, for example, recombinant cells express genes that are not found within the native (non-recombinant) form of the cell, or express native genes at different levels or under different conditions than found in nature. The term “recombinant” is synonymous with “genetically modified” and “transgenic”.

Recover: The terms “recover” or “recovery” means the removal of a polypeptide from at least one fermentation broth component selected from the list of a cell, a nucleic acid, or other specified material, e.g., recovery of the polypeptide from the whole fermentation broth, or from the cell-free fermentation broth, by polypeptide crystal harvest, by filtration, e.g., depth filtration (by use of filter aids or packed filter medias, cloth filtration in chamber filters, rotary-drum filtration, drum filtration, rotary vacuum-drum filters, candle filters, horizontal leaf filters or similar, using sheed or pad filtration in framed or modular setups) or membrane filtration (using sheet filtration, module filtration, candle filtration, microfiltration, ultrafiltration in either cross flow, dynamic cross flow or dead end operation), or by centrifugation (using decanter centrifuges, disc stack centrifuges, hyrdo cyclones or similar), or by precipitating the polypeptide and using relevant solid-liquid separation methods to harvest the polypeptide from the broth media by use of classification separation by particle sizes. Recovery encompasses isolation and/or purification of the polypeptide.

Sequence identity: The relatedness between two amino acid sequences or between two nucleotide sequences is described by the parameter “sequence identity”.

For purposes of the present invention, the sequence identity between two amino acid sequences is determined as the output of “longest identity” using the Needleman-Wunsch algorithm (Needleman and Wunsch, 1970, J. Mol. Biol. 48:443-453) as implemented in the Needle program of the EMBOSS package (EMBOSS: The European Molecular Biology Open Software Suite, Rice et al., 2000, Trends Genet. 16:276-277), preferably version 6.6.0 or later. The parameters used are a gap open penalty of 10, a gap extension penalty of 0.5, and the EBLOSUM62 (EMBOSS version of BLOSUM62) substitution matrix. In order for the Needle program to report the longest identity, the-nobrief option must be specified in the command line. The output of Needle labeled “longest identity” is calculated as follows:

(Identical Residues×100)/(Length of Alignment−Total Number of Gaps in Alignment)

For purposes of the present invention, the sequence identity between two polynucleotide sequences is determined as the output of “longest identity” using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 6.6.0 or later. The parameters used are a gap open penalty of 10, a gap extension penalty of 0.5, and the EDNAFULL (EMBOSS version of NCBI NUC4.4) substitution matrix. In order for the Needle program to report the longest identity, the nobrief option must be specified in the command line. The output of Needle labeled “longest identity” is calculated as follows:

(Identical Deoxyribonucleotides×100)/(Length of Alignment−Total Number of Gaps in Alignment)

Signal Peptide: A “signal peptide” is a sequence of amino acids attached to the N-terminal portion of a protein, which facilitates the secretion of the protein outside the cell. The mature form of an extracellular protein lacks the signal peptide, which is cleaved off during the secretion process.

Subsequence: The term “subsequence” means a polynucleotide having one or more nucleotides absent from the 5′ and/or 3′ end of a mature polypeptide coding sequence; wherein the subsequence encodes a fragment having lysozyme activity.

Variant: The term “variant” means a polypeptide having lysozyme activity comprising a substitution, an insertion (including extension), and/or a deletion (e.g., truncation), at one or more positions. A substitution means replacement of the amino acid occupying a position with a different amino acid; a deletion means removal of the amino acid occupying a position; and an insertion means adding 1-5 amino acids (e.g., 1-3 amino acids, in particular, 1 amino acid) adjacent to and immediately following the amino acid occupying a position.

Wild-type: The term “wild-type” in reference to an amino acid sequence or nucleic acid sequence means that the amino acid sequence or nucleic acid sequence is a native or naturally occurring sequence. As used herein, the term “naturally-occurring” refers to anything (e.g., proteins, amino acids, or nucleic acid sequences) that is found in nature. Conversely, the term “non-naturally occurring” refers to anything that is not found in nature (e.g., recombinant nucleic acids and protein sequences produced in the laboratory or modification of the wild-type sequence).

### Conventions for Designation of Polypeptide of the Invention

For purposes of the present invention, the polypeptide disclosed in SEQ ID NO: 1 is used to determine the corresponding amino acid positions in another lysozyme. The amino acid sequence of another lysozyme is aligned with the polypeptide disclosed in SEQ ID NO: 1, and based on the alignment, the amino acid position number corresponding to any amino acid residue in the polypeptide disclosed in SEQ ID NO: 1 is determined using the Needleman-Wunsch algorithm (Needleman and Wunsch, 1970, J. Mol. Biol. 48:443-453) as implemented in the Needle program of the EMBOSS package (EMBOSS: The European Molecular Biology Open Software Suite, Rice et al., 2000, Trends Genet. 16:276-277), preferably version 5.0.0 or later.

The parameters used are gap open penalty of 10, gap extension penalty of 0.5, and the EBLOSUM62 (EMBOSS version of BLOSUM62) substitution matrix.

In describing the polypeptide of the present invention, the nomenclature described below is adapted for ease of reference. The accepted IUPAC single letter or three letter amino acid abbreviation is employed.

Substitutions. For an amino acid substitution, the following nomenclature is used: Original amino acid, position, substituted amino acid. Accordingly, the substitution of threonine at position 226 with alanine is designated as “Thr226Ala” or “T226A”. Multiple mutations are separated by addition marks (“+”), e.g., “Gly205Arg+Ser411Phe” or “G205R+S411F”, representing substitutions at positions 205 and 411 of glycine (G) with arginine (R) and serine(S) with phenylalanine (F), respectively.

Multiple alterations. Variants comprising multiple alterations are separated by addition marks (“+”), e.g., “Arg170Tyr+Gly195Glu” or “R170Y+G195E” representing a substitution of arginine and glycine at positions 170 and 195 with tyrosine and glutamic acid, respectively.

Different alterations. Where different alterations can be introduced at a position, the different alterations are separated by a comma, e.g., “Arg170Tyr, Glu” represents a substitution of arginine at position 170 with tyrosine or glutamic acid. Thus, “Tyr167Gly, Ala+Arg170Gly,Ala” designates the following:
“Tyr167Gly+Arg170Gly”, “Tyr167Gly+Arg170Ala”, “Tyr167Ala+Arg170Gly”, and “Tyr167Ala+Arg170Ala”.

The present invention relates to a polypeptide having lysozyme activity, comprising a substitution at one or more positions corresponding to positions 1, 2, 6, 9, 10, 13, 14, 20, 36, 37, 40, 41, 63, 65, 101, 103, 118, 122, 125, 126, 129 131, 143, 153, 155, 166 174, 175, 176, 188 and 202 of SEQ ID NO:1, wherein the polypeptide has at least 60%, e.g., at least 65%, at least 70%, at least 75%, at least 80%, at least 85%, at least 90%, at least 95%, at least 96%, at least 97%, at least 98%, or at least 99% sequence identity, but less than 100% sequence identity, to SEQ ID NO: 1.

The polypeptide may further comprise an extension of one or more amino acids at the N-terminal and/or C-terminal ends.

Alternatively, the polypeptide may further comprise a truncation of one or more amino acids at the N-terminal and/or C-terminal ends.

### Polypeptides of the Invention

In another embodiment, the polypeptide has at least 60%, e.g., at least 65%, at least 70%, at least 75%, at least 80%, at least 85%, at least 90%, at least 91%, at least 92%, at least 93%, at least 94%, at least 95%, such as at least 96%, at least 97%, at least 98%, or at least 99%, but less than 100%, sequence identity to SEQ ID NO:1, or amino acids 20 to 227 of SEQ ID NO: 2.

In one aspect, the polypeptide of the present invention comprises 1-20 alterations, e.g., 1-10, such as 1, 2, 3, 4, 5, 6, 7, 8, 9 or 10 alterations.

In another aspect, the polypeptide comprises a substitution at one or more positions corresponding to positions 1, 2, 6, 9, 10, 13, 14, 20, 36, 37, 40, 41, 63, 65, 101, 103, 118, 122, 125, 126, 129 131, 143, 153, 155, 166 174, 175, 176, 188 and 202 of SEQ ID NO:1. In another aspect, the polypeptide comprises substitutions at two positions corresponding to any of positions 10, 13, 14, 143, 166, and 176 of SEQ ID NO:1. In another aspect, the polypeptide comprises substitutions at three positions corresponding to any of positions 10, 13, 14, 37, 101, 153, 166, 176, 202 of SEQ ID NO:1. In another aspect, the polypeptide comprises substitutions at four positions corresponding to any of positions 2, 6, 9, 10, 13, 14, 20, 36, 37, 40, 63, 65, 101, 118, 122, 125, 129, 131, 166, 175, 176 and 202 of SEQ ID NO:1. In another aspect, a polypeptide comprises substitutions at five positions corresponding to any of positions 1, 10, 13, 14, 118, 122, 166, 176 of SEQ ID NO:1.

In another aspect, the polypeptide comprises or consists of a substitution at a position corresponding to position 1 of SEQ ID NO: 1. In another aspect, the amino acid at a position corresponding to position 1 of SEQ ID NO: 1 is substituted with Ala, Asn, Asp, Cys, Gln, Glu, Gly, His, Ile, Leu, Lys, Met, Phe, Pro, Ser, Thr, Trp, Tyr, or Val, preferably with Thr or Gln. In another aspect, the polypeptide comprises or consists of the substitution R1T of SEQ ID NO:1. In yet another aspect, the polypeptide is SEQ ID NO: 1 substituted at position 1, namely comprising or consisting of the substitution at position 1, such as R1Q. Substitution at position 1 of SEQ ID NO: 1, as shown in Tables 2 and 4, may with some substitutions, negatively impact thermostability, it provides however, the advantage of preventing glycation of the muramidase. In some embodiments of substitutions at position 1 of SEQ ID NO:1, both an increased thermostability is observed and a reduction in glycation is observed.

In another aspect, the polypeptide comprises or consists of a substitution at a position corresponding to position 2 of SEQ ID NO: 1. In another aspect, the amino acid at a position corresponding to position 2 of SEQ ID NO: 1 is substituted with Ala, Arg, Asn, Asp, Cys, Gln, Glu, Gly, His, Leu, Lys, Met, Phe, Pro, Ser, Thr, Trp, Tyr, or Val, preferably with Val. In yet another aspect, the polypeptide is SEQ ID NO: 1 substituted at position 2, namely comprising or consisting of the substitution at position 2, such as 12V.

In another aspect, the polypeptide comprises or consists of a substitution at a position corresponding to position 6 of SEQ ID NO: 1. In another aspect, the amino acid at a position corresponding to position 6 of SEQ ID NO: 1 is substituted with Ala, Arg, Asn, Cys, Gln, Glu, Gly, His, Ile, Leu, Lys, Met, Phe, Pro, Ser, Thr, Trp, Tyr, or Val, preferably with Ser. In yet another aspect, the polypeptide is SEQ ID NO: 1 substituted at position 6, namely comprising or consisting of the substitution at position 6, such as D6S.

In another aspect, the polypeptide comprises or consists of a substitution at a position corresponding to position 9 of SEQ ID NO: 1. In another aspect, the amino acid at a position corresponding to position 9 of SEQ ID NO: 1 is substituted with Ala, Arg, Asn, Asp, Cys, Gln, Glu, His, Ile, Leu, Lys, Met, Phe, Pro, Ser, Thr, Trp, Tyr, or Val, preferably with Pro. In yet another aspect, the polypeptide is SEQ ID NO: 1 substituted at position 9, namely comprising or consisting of the substitution at position 9, such as G9P.

In another aspect, the polypeptide comprises or consists of a substitution at a position corresponding to position 10 of SEQ ID NO: 1. In another aspect, the amino acid at a position corresponding to position 10 of SEQ ID NO: 1 is substituted with Ala, Arg, Asn, Asp, Cys, Gln, Glu, Gly, His, Ile, Leu, Lys, Met, Phe, Pro, Ser, Thr, Tyr, or Val, preferably with Ala, Asp or Arg. In another aspect, the polypeptide comprises or consists of the substitution W10A, W10D or W10R of SEQ ID NO:1.

In another aspect, the polypeptide comprises or consists of a substitution at a position corresponding to position 13 of SEQ ID NO: 1. In another aspect, the amino acid at a position corresponding to position 13 of SEQ ID NO: 1 is substituted with Ala, Arg, Asn, Asp, Cys, Gln, Glu, Gly, His, Ile, Leu, Lys, Met, Phe, Pro, Ser, Trp, Tyr, or Val, preferably with Asn. In yet another aspect, the polypeptide is SEQ ID NO: 1 substituted at position 13, namely comprising or consisting of the substitution at position 13, such as T13N.

In another aspect, the polypeptide comprises or consists of a substitution at a position corresponding to position 14 of SEQ ID NO: 1. In another aspect, the amino acid at a position corresponding to position 14 of SEQ ID NO: 1 is substituted with Ala, Arg, Asn, Asp, Cys, Gln, Glu, Gly, His, Ile, Leu, Lys, Met, Phe, Pro, Ser, Trp, Tyr, or Val, preferably with Val. In another aspect, the polypeptide comprises or consists of the substitution T14V of SEQ ID NO:1.

In another aspect, the polypeptide comprises or consists of a substitution at a position corresponding to position 20 of SEQ ID NO: 1. In another aspect, the amino acid at a position corresponding to position 20 of SEQ ID NO: 1 is substituted with Ala, Arg, Asn, Asp, Cys, Gln, Glu, Gly, His, Ile, Leu, Lys, Met, Phe, Pro, Ser, Thr, Trp, or Val, preferably with Trp. In another aspect, the polypeptide comprises or consists of the substitution Y20W of SEQ ID NO:1.

In another aspect, the polypeptide comprises or consists of a substitution at a position corresponding to position 36 of SEQ ID NO: 1. In another aspect, the amino acid at a position corresponding to position 36 of SEQ ID NO: 1 is substituted with Ala, Arg, Asn, Asp, Cys, Gln, Glu, Gly, His, Ile, Leu, Lys, Met, Phe, Pro, Ser, Trp, Tyr, or Val, preferably with Glu. In another aspect, the polypeptide comprises or consists of the substitution T36E of SEQ ID NO:1.

In another aspect, the polypeptide comprises or consists of a substitution at a position corresponding to position 37 of SEQ ID NO: 1. In another aspect, the amino acid at a position corresponding to position 37 of SEQ ID NO: 1 is substituted with Ala, Arg, Asn, Asp, Cys, Gln, Glu, Gly, His, Ile, Leu, Lys, Met, Pro, Ser, Thr, Trp, Tyr, or Val, preferably with Asn or Tyr. In another aspect, the polypeptide comprises or consists of the substitution F37N or F37Y of SEQ ID NO: 1.

In another aspect, the polypeptide comprises or consists of a substitution at a position corresponding to position 40 of SEQ ID NO: 1. In another aspect, the amino acid at a position corresponding to position 40 of SEQ ID NO: 1 is substituted with Ala, Arg, Asn, Asp, Cys, Gln, Glu, Gly, His, Ile, Leu, Lys, Met, Phe, Pro, Thr, Trp, Tyr, or Val, preferably with Pro. In another aspect, the polypeptide comprises or consists of the substitution S40P of SEQ ID NO:1.

In another aspect, the polypeptide comprises or consists of a substitution at a position corresponding to position 41 of SEQ ID NO: 1. In another aspect, the amino acid at a position corresponding to position 41 of SEQ ID NO: 1 is substituted with Arg, Asn, Asp, Cys, Gln, Glu, Gly, His, Ile, Leu, Lys, Met, Phe, Pro, Ser, Thr, Trp, Tyr, or Val, preferably with Tyr. In another aspect, the polypeptide comprises or consists of the substitution A41Y of SEQ ID NO: 1.

In another aspect, the polypeptide comprises or consists of a substitution at a position corresponding to position 63 of SEQ ID NO: 1. In another aspect, the amino acid at a position corresponding to position 63 of SEQ ID NO: 1 is substituted with Ala, Arg, Asn, Asp, Cys, Glu, Gly, His, Ile, Leu, Lys, Met, Phe, Pro, Ser, Thr, Trp, Tyr, or Val, preferably with Arg. In another aspect, the polypeptide comprises or consists of the substitution Q63R of SEQ ID NO:1.

In another aspect, the polypeptide comprises or consists of a substitution at a position corresponding to position 65 of SEQ ID NO: 1. In another aspect, the amino acid at a position corresponding to position 65 of SEQ ID NO: 1 is substituted with Arg, Asn, Asp, Cys, Gln, Glu, Gly, His, Ile, Leu, Lys, Met, Phe, Pro, Ser, Thr, Trp, Tyr, or Val, preferably with Asp. In another aspect, the polypeptide comprises or consists of the substitution A65D of SEQ ID NO:1.

In another aspect, the polypeptide comprises or consists of a substitution at a position corresponding to position 101 of SEQ ID NO: 1. In another aspect, the amino acid at a position corresponding to position 101 of SEQ ID NO: 1 is substituted with Ala, Arg, Asp, Cys, Gln, Glu, Gly, His, Ile, Leu, Lys, Met, Phe, Pro, Ser, Thr, Trp, Tyr, or Val, preferably with Ser. In another aspect, the polypeptide comprises or consists of the substitution N101S of SEQ ID NO:1.

In another aspect, the polypeptide comprises or consists of a substitution at a position corresponding to position 103 of SEQ ID NO: 1. In another aspect, the amino acid at a position corresponding to position 103 of SEQ ID NO: 1 is substituted with Arg, Asn, Asp, Cys, Gln, Glu, Gly, His, Ile, Leu, Lys, Met, Phe, Pro, Ser, Thr, Trp, Tyr, or Val, preferably with Ser. In another aspect, the polypeptide comprises or consists of the substitution A103S of SEQ ID NO: 1.

In another aspect, the polypeptide comprises or consists of a substitution at a position corresponding to position 118 of SEQ ID NO: 1. In another aspect, the amino acid at a position corresponding to position 118 of SEQ ID NO: 1 is substituted with Ala, Arg, Asn, Asp, Cys, Gln, Gly, His, Ile, Leu, Lys, Met, Phe, Pro, Ser, Thr, Trp, Tyr, or Val, preferably with Arg. In another aspect, the polypeptide comprises or consists of the substitution E118R of SEQ ID NO: 1.

In another aspect, the polypeptide comprises or consists of a substitution at a position corresponding to position 122 of SEQ ID NO: 1. In another aspect, the amino acid at a position corresponding to position 122 of SEQ ID NO: 1 is substituted with Ala, Arg, Asn, Asp, Cys, Gln, Glu, Gly, His, Ile, Leu, Lys, Met, Phe, Pro, Ser, Trp, Tyr, or Val, preferably with Pro or Asp. In another aspect, the polypeptide comprises or consists of the substitution T122P or T122D of SEQ ID NO: 1.

In another aspect, the polypeptide comprises or consists of a substitution at a position corresponding to position 125 of SEQ ID NO: 1. In another aspect, the amino acid at a position corresponding to position 125 of SEQ ID NO: 1 is substituted with Ala, Arg, Asn, Asp, Cys, Gln, Glu, Gly, Ile, Leu, Lys, Met, Phe, Pro, Ser, Thr, Trp, Tyr, or Val, preferably with Pro or Lys. In another aspect, the polypeptide comprises or consists of the substitution H125P or H125K of SEQ ID NO: 1.

In another aspect, the polypeptide comprises or consists of a substitution at a position corresponding to position 126 of SEQ ID NO: 1. In another aspect, the amino acid at a position corresponding to position 126 of SEQ ID NO: 1 is substituted with Ala, Arg, Asn, Asp, Cys, Gln, Glu, His, Ile, Leu, Lys, Met, Phe, Pro, Ser, Thr, Trp, Tyr, or Val, preferably with Asn or Pro. In another aspect, the polypeptide comprises or consists of the substitution G126N or G126P of SEQ ID NO: 1.

In another aspect, the polypeptide comprises or consists of a substitution at a position corresponding to position 129 of SEQ ID NO: 1. In another aspect, the amino acid at a position corresponding to position 129 of SEQ ID NO: 1 is substituted with Ala, Arg, Asn, Asp, Cys, Gln, Glu, Gly, His, Ile, Leu, Lys, Met, Phe, Pro, Thr, Trp, Tyr, or Val, preferably with Gly. In another aspect, the polypeptide comprises or consists of the substitution S129G of SEQ ID NO:1.

In another aspect, the polypeptide comprises or consists of a substitution at a position corresponding to position 131 of SEQ ID NO: 1. In another aspect, the amino acid at a position corresponding to position 131 of SEQ ID NO: 1 is substituted with Ala, Arg, Asn, Asp, Cys, Gln, Glu, Gly, His, Ile, Leu, Lys, Met, Phe, Pro, Ser, Thr, Trp or Tyr, preferably with Cys or Tyr. In another aspect, the polypeptide comprises or consists of the substitution V131C or W131Y of SEQ ID NO:1.

In another aspect, the polypeptide comprises or consists of a substitution at a position corresponding to position 143 of SEQ ID NO: 1. In another aspect, the amino acid at a position corresponding to position 143 of SEQ ID NO: 1 is substituted with Ala, Arg, Asn, Asp, Cys, Glu, Gly, His, Ile, Leu, Lys, Met, Phe, Pro, Ser, Thr, Trp, Tyr, or Val, preferably with Asn. In another aspect, the polypeptide comprises or consists of the substitution Q143N of SEQ ID NO:1.

In another aspect, the polypeptide comprises or consists of a substitution at a position corresponding to position 153 of SEQ ID NO: 1. In another aspect, the amino acid at a position corresponding to position 153 of SEQ ID NO: 1 is substituted with Ala, Arg, Asp, Cys, Gln, Glu, Gly, His, Ile, Leu, Lys, Met, Phe, Pro, Ser, Thr, Trp, Tyr, or Val, preferably with Ser. In another aspect, the polypeptide comprises or consists of the substitution N153S of SEQ ID NO:1.

In another aspect, the polypeptide comprises or consists of a substitution at a position corresponding to position 155 of SEQ ID NO: 1. In another aspect, the amino acid at a position corresponding to position 155 of SEQ ID NO: 1 is substituted with Ala, Arg, Asn, Asp, Gln, Glu, Gly, His, Ile, Leu, Lys, Met, Phe, Pro, Ser, Thr, Trp, Tyr, or Val, preferably with Trp. In another aspect, the polypeptide comprises or consists of the substitution C155W of SEQ ID NO:1.

In another aspect, the polypeptide comprises or consists of a substitution at a position corresponding to position 166 of SEQ ID NO: 1. In another aspect, the amino acid at a position corresponding to position 166 of SEQ ID NO: 1 is substituted with Ala, Arg, Asn, Asp, Cys, Gln, Glu, Gly, His, Ile, Leu, Lys, Met, Phe, Pro, Ser, Thr, Trp, or Tyr, preferably with Pro. In another aspect, the polypeptide comprises or consists of the substitution V166P of SEQ ID NO:1.

In another aspect, the polypeptide comprises or consists of a substitution at a position corresponding to position 174 of SEQ ID NO: 1. In another aspect, the amino acid at a position corresponding to position 174 of SEQ ID NO: 1 is substituted with Ala, Arg, Asn, Asp, Cys, Gln, Glu, His, Ile, Leu, Lys, Met, Phe, Pro, Ser, Thr, Trp, Tyr, or Val, preferably with Pro. In another aspect, the polypeptide comprises or consists of the substitution G174P of SEQ ID NO:1.

In another aspect, the polypeptide comprises or consists of a substitution at a position corresponding to position 175 of SEQ ID NO: 1. In another aspect, the amino acid at a position corresponding to position 175 of SEQ ID NO: 1 is substituted with Ala, Arg, Asn, Asp, Cys, Gln, Glu, Gly, His, Ile, Leu, Lys, Met, Pro, Ser, Thr, Trp, Tyr, or Val, preferably with Ser or Asn. In another aspect, the polypeptide comprises or consists of the substitution F175S or F175N of SEQ ID NO: 1.

In another aspect, the polypeptide comprises or consists of a substitution at a position corresponding to position 176 of SEQ ID NO: 1. In another aspect, the amino acid at a position corresponding to position 176 of SEQ ID NO: 1 is substituted with Ala, Arg, Asn, Asp, Cys, Gln, Glu, Gly, His, Ile, Leu, Lys, Met, Phe, Pro, Ser, Thr, Trp, or Val, preferably with Trp. In another aspect, the polypeptide comprises or consists of the substitution Y176W of SEQ ID NO:1.

In another aspect, the polypeptide comprises or consists of a substitution at a position corresponding to position 188 of SEQ ID NO: 1. In another aspect, the amino acid at a position corresponding to position 188 of SEQ ID NO: 1 is substituted with Ala, Arg, Asn, Asp, Cys, Gln, Glu, His, Ile, Leu, Lys, Met, Phe, Pro, Ser, Thr, Trp, Tyr, or Val, preferably with Pro. In another aspect, the polypeptide comprises or consists of the substitution G188P of SEQ ID NO:1.

In another aspect, the polypeptide comprises or consists of a substitution at a position corresponding to position 202 of SEQ ID NO: 1. In another aspect, the amino acid at a position corresponding to position 202 of SEQ ID NO: 1 is substituted with Ala, Asn, Asp, Cys, Gln, Glu, Gly, His, Ile, Leu, Lys, Met, Phe, Pro, Ser, Thr, Trp, Tyr, or Val, preferably with Glu. In another aspect, the polypeptide comprises or consists of the substitution R202E of SEQ ID NO: 1.

In another aspect, the polypeptide comprises or consists of substitutions at positions corresponding to positions 10 and 143 of SEQ ID NO: 1, such as those described above. In another aspect, the polypeptide comprises or consists of the substitutions W10D and Q143E of SEQ ID NO:1 of SEQ ID NO: 1. In another aspect, the amino acid at a position corresponding to position 10 is substituted preferably with Arg.

In another aspect, the polypeptide comprises or consists of substitutions at positions corresponding to positions 13 and 176 of SEQ ID NO: 1. In another aspect, the polypeptide comprises or consists of the substitutions T13N and Y176W of SEQ ID NO: 1.

In another aspect, the polypeptide comprises or consists of substitutions at positions corresponding to positions 10 and 14 of SEQ ID NO: 1, such as those described above. In another aspect, the amino acid at a position corresponding to position 10 is substituted preferably with Ala. In another aspect, the polypeptide comprises or consists of the substitutions W10A and T14V of SEQ ID NO: 1.

In another aspect, the polypeptide comprises or consists of substitutions at positions corresponding to positions 10 and 176 of SEQ ID NO: 1, such as those described above. In another aspect, the polypeptide comprises or consists of the substitutions W10R and Y176W of SEQ ID NO: 1.

In another aspect, the polypeptide comprises or consists of substitutions at positions corresponding to positions 10 and 166 of SEQ ID NO: 1. In another aspect, the polypeptide comprises or consists of the substitutions W10R and V166P of SEQ ID NO: 1.

In another aspect, the polypeptide comprises or consists of substitutions at positions corresponding to positions 166 and 176 of SEQ ID NO: 1. In another aspect, the polypeptide comprises or consists of the substitutions V166P and Y176W of SEQ ID NO: 1.

In another aspect, the polypeptide comprises or consists of substitutions at positions corresponding to positions 10, 13, and 37 of SEQ ID NO: 1, such as those described above. In another aspect, the amino acid at a position corresponding to position 10 is substituted preferably with Arg and the amino acid at a position corresponding to position 37 is substituted preferably with Tyr.

In another aspect, the polypeptide comprises or consists of substitutions at positions corresponding to positions 13, 14, and 37 of SEQ ID NO: 1, such as those described above. In another aspect, the amino acid at a position corresponding to position 37 is substituted preferably with Tyr.

In another aspect, the polypeptide comprises or consists of substitutions at positions corresponding to positions 10, 13, and 101 of SEQ ID NO: 1, such as those described above. In another aspect, the amino acid at a position corresponding to position 10 is substituted preferably with Arg.

In another aspect, the polypeptide comprises or consists of substitutions at positions corresponding to positions 10, 166, and 176 of SEQ ID NO: 1, such as those described above. In another aspect, the amino acid at a position corresponding to position 10 is substituted preferably with Asp.

In another aspect, the polypeptide comprises or consists of substitutions at positions corresponding to positions 10, 13, and 14 of SEQ ID NO: 1, such as those described above. In another aspect, the amino acid at a position corresponding to position 10 is substituted preferably with Ala.

In another aspect, the polypeptide comprises or consists of substitutions at positions corresponding to positions 10, 14, and 101 of SEQ ID NO: 1, such as those described above. In another aspect, the amino acid at a position corresponding to position 10 is substituted preferably with Arg.

In another aspect, the polypeptide comprises or consists of substitutions at positions corresponding to positions 10, 101, and 153 of SEQ ID NO: 1, such as those described above. In another aspect, the amino acid at a position corresponding to position 10 is substituted preferably with Asp.

In another aspect, the polypeptide comprises or consists of substitutions at positions corresponding to positions 10, 14, and 37 of SEQ ID NO: 1, such as those described above. In another aspect, the amino acid at a position corresponding to position 10 is substituted preferably with Ala and the amino acid at a position corresponding to position 37 is substituted preferably with Tyr.

In another aspect, the polypeptide comprises or consists of substitutions at positions corresponding to positions 13, 14, and 202 of SEQ ID NO: 1, such as those described above.

In another aspect, the polypeptide comprises or consists of substitutions at positions corresponding to positions 10, 14, and 153 of SEQ ID NO: 1, such as those described above. In another aspect, the amino acid at a position corresponding to position 10 of SEQ ID NO: 1 is substituted preferably with Ala.

In another aspect, the polypeptide comprises or consists of substitutions at positions corresponding to positions 13, 166, and 176 of SEQ ID NO: 1, such as those described above.

In another aspect, the polypeptide comprises or consists of substitutions at positions corresponding to positions 14, 166, and 176 of SEQ ID NO: 1, such as those described above.

In another aspect, the polypeptide comprises or consists of substitutions at positions corresponding to positions 10, 166, and 176 of SEQ ID NO: 1, such as those described above. In another aspect, the amino acid at a position corresponding to position 10 is substituted preferably with Ala.

In another aspect, the polypeptide comprises or consists of substitutions at positions corresponding to positions 10, 166, and 176 of SEQ ID NO: 1, such as those described above. In another aspect, the amino acid at a position corresponding to position 10 of SEQ ID NO: 1 is substituted preferably with Asp.

In another aspect, the polypeptide comprises or consists of substitutions at positions corresponding to positions 10, 14, 166, and 176 of SEQ ID NO: 1, such as those described above.

In another aspect, the polypeptide comprises or consists of substitutions at positions corresponding to positions 10, 101, 166, and 176 of SEQ ID NO: 1, such as those described above. In another aspect, the amino acid at a position corresponding to position 10 of SEQ ID NO: 1 is substituted preferably with Asp.

In another aspect, the polypeptide comprises or consists of substitutions at positions corresponding to positions 10, 14, 118, and 122 of SEQ ID NO: 1, such as those described above. In another aspect, the amino acid at a position corresponding to position 10 of SEQ ID NO: 1 is substituted preferably with Asp. In another aspect, the amino acid at a position corresponding to position 122 of SEQ ID NO: 1 is substituted preferably with Asp.

In another aspect, the polypeptide comprises or consists of substitutions at positions corresponding to positions 10, 166, 176, and 202 of SEQ ID NO: 1, such as those described above. In another aspect, the amino acid at a position corresponding to position 10 is substituted preferably with Asp.

In another aspect, the polypeptide comprises or consists of substitutions at positions corresponding to positions 10, 13, 118, and 122 of SEQ ID NO: 1, such as those described above. In another aspect, the amino acid at a position corresponding to position 10 of SEQ ID NO: 1 is substituted preferably with Ala. In another aspect, the amino acid at a position corresponding to position 122 of SEQ ID NO: 1 is substituted preferably with Asp.

In another aspect, the polypeptide comprises or consists of substitutions at positions corresponding to positions 37, 166, 176, and 202 of SEQ ID NO: 1, such as those described above. In another aspect, the amino acid at a position corresponding to position 37 of SEQ ID NO: 1 is substituted preferably with Tyr.

In another aspect, the polypeptide comprises or consists of substitutions at positions corresponding to positions 10, 118, 122, and 202 of SEQ ID NO: 1, such as those described above. In another aspect, the amino acid at a position corresponding to position 10 of SEQ ID NO: 1 is substituted preferably with Ala. In another aspect, the amino acid at a position corresponding to position 122 of SEQ ID NO: 1 is substituted preferably with Asp.

In another aspect, the polypeptide comprises or consists of substitutions at positions corresponding to positions 6, 10, 101, and 166 of SEQ ID NO: 1, such as those described above. In another aspect, the amino acid at a position corresponding to position 10 of SEQ ID NO: 1 is substituted preferably with Asp.

In another aspect, the polypeptide comprises or consists of substitutions at positions corresponding to positions 6, 9, 10, and 101 of SEQ ID NO: 1, such as those described above. In another aspect, the amino acid at a position corresponding to position 10 of SEQ ID NO: 1 is substituted preferably with Asp.

In another aspect, the polypeptide comprises or consists of substitutions at positions corresponding to positions 6, 10, 36, and 101 of SEQ ID NO: 1, such as those described above. In another aspect, the amino acid at a position corresponding to position 10 of SEQ ID NO: 1 is substituted preferably with Asp.

In another aspect, the polypeptide comprises or consists of substitutions at positions corresponding to positions 9, 10, 101, and 175 of SEQ ID NO: 1, such as those described above. In another aspect, the amino acid at a position corresponding to position 10 of SEQ ID NO: 1 is substituted preferably with Asp. In another aspect, the amino acid at a position corresponding to position 175 is substituted preferably with Asn.

In another aspect, the polypeptide comprises or consists of substitutions at positions corresponding to positions 9, 10, 101, and 129 of SEQ ID NO: 1, such as those described above. In another aspect, the amino acid at a position corresponding to position 10 of SEQ ID NO: 1 is substituted preferably with Asp.

In another aspect, the polypeptide comprises or consists of substitutions at positions corresponding to positions 10, 36, 37, and 101 of SEQ ID NO: 1, such as those described above.

In another aspect, the amino acid at a position corresponding to position 10 of SEQ ID NO: 1 is substituted preferably with Asp. In another aspect, the amino acid at a position corresponding to position 37 of SEQ ID NO: 1 is substituted preferably with Glu.

In another aspect, the polypeptide comprises or consists of substitutions at positions corresponding to positions 10, 40, 101, and 175 of SEQ ID NO: 1, such as those described above. In another aspect, the amino acid at a position corresponding to position 10 of SEQ ID NO: 1 is substituted preferably with Asp. In another aspect, the amino acid at a position corresponding to position 175 of SEQ ID NO: 1 is substituted preferably with Asn.

In another aspect, the polypeptide comprises or consists of substitutions at positions corresponding to positions 10, 20, 101, and 175 of SEQ ID NO: 1, such as those described above. In another aspect, the amino acid at a position corresponding to position 10 of SEQ ID NO: 1 is substituted preferably with Asp. In another aspect, the amino acid at a position corresponding to position 175 of SEQ ID NO: 1 is substituted preferably with Asn.

In another aspect, the polypeptide comprises or consists of substitutions at positions corresponding to positions 2, 9, 10, and 101 of SEQ ID NO: 1, such as those described above. In another aspect, the amino acid at a position corresponding to position 10 of SEQ ID NO: 1 is substituted preferably with Asp.

In another aspect, the polypeptide comprises or consists of substitutions at positions corresponding to positions 10, 63, 65, and 101 of SEQ ID NO: 1, such as those described above. In another aspect, the amino acid at a position corresponding to position 10 of SEQ ID NO: 1 is substituted preferably with Asp.

In another aspect, the polypeptide comprises or consists of substitutions at positions corresponding to positions 10, 101, 125, and 131 of SEQ ID NO: 1, such as those described above. In another aspect, the amino acid at a position corresponding to position 10 of SEQ ID NO: 1 is substituted preferably with Asp. In another aspect, the amino acid at a position corresponding to position 125 of SEQ ID NO: 1 is substituted preferably with Lys. In another aspect, the amino acid at a position corresponding to position 131 of SEQ ID NO: 1 is substituted preferably with Tyr.

In another aspect, the polypeptide comprises or consists of substitutions at positions corresponding to positions 13, 14, 166, and 176 of SEQ ID NO: 1, such as those described above.

In another aspect, the polypeptide comprises or consists of substitutions at positions corresponding to positions 10, 13, 166, and 176 of SEQ ID NO: 1, such as those described above. In another aspect, the amino acid at a position corresponding to position 10 of SEQ ID NO: 1 is substituted preferably with Ala.

In another aspect, the polypeptide comprises or consists of substitutions at positions corresponding to positions 37, 166, 176, and 202 of SEQ ID NO: 1, such as those described above. In another aspect, the amino acid at a position corresponding to position 37 of SEQ ID NO: 1 is substituted preferably with Tyr.

In another aspect, the polypeptide comprises or consists of substitutions at positions corresponding to positions 10, 166, 176, and 202 of SEQ ID NO: 1, such as those described above. In another aspect, the amino acid at a position corresponding to position 10 of SEQ ID NO: 1 is substituted preferably with Ala.

In another aspect, the polypeptide comprises or consists of substitutions at positions corresponding to positions 10, 37, 166, and 176 of SEQ ID NO: 1, such as those described above. In another aspect, the amino acid at a position corresponding to position 10 of SEQ ID NO: 1 is substituted preferably with Ala. In another aspect, the amino acid at a position corresponding to position 37 of SEQ ID NO: 1 is substituted preferably with Tyr.

In another aspect, the polypeptide comprises or consists of substitutions at positions corresponding to positions 10, 118, 122, 166 and 176 of SEQ ID NO: 1, such as those described above.

In another aspect, the polypeptide comprises or consists of substitutions at positions corresponding to positions 10, 13, 14, 166 and 176 of SEQ ID NO: 1, such as those described above.

In another aspect, the polypeptide comprises or consists of substitutions at positions corresponding to positions 10, 13, 122, 166 and 176 of SEQ ID NO: 1, such as those described above.

In another aspect, the polypeptide comprises or consists of substitutions at positions corresponding to positions 1, 10, 13, 14, 166 and 176 of SEQ ID NO: 1, such as those described above.

In another aspect, the polypeptide comprises or consists of one or more substitutions selected from the group consisting of R1T, R1Q, W10A, W10D, W10R, T14V, F37N, F37Y, A41Y, A103S, E118R, T122P, H125P, G126N, G126P, V131C, Q143N, N153S, C155W, G174P, F175S, G188P, R202E of SEQ ID NO: 1.

In another aspect, the polypeptide comprises or consists of the substitutions W10D+Q143E of SEQ ID NO: 1.

In another aspect, the polypeptide comprises or consists of the substitutions V166P+Y176W of SEQ ID NO: 1.

In another aspect, the polypeptide comprises or consists of the substitutions T13N+Y176W of SEQ ID NO: 1.

In another aspect, the polypeptide comprises or consists of the W10A+T14V of SEQ ID NO: 1.

In another aspect, the polypeptide comprises or consists of the substitutions W10D+T14V of SEQ ID NO: 1.

In another aspect, the polypeptide comprises or consists of the substitutions W10R+Y176W of SEQ ID NO: 1.

In another aspect, the polypeptide comprises or consists of the substitutions W10R+V166P of SEQ ID NO: 1.

In another aspect, the polypeptide comprises or consists of the W10R+T13N+F37Y of SEQ ID NO: 1.

In another aspect, the polypeptide comprises or consists of the T13N+T14V+F37Y of SEQ ID NO: 1. In another aspect, the polypeptide comprises or consists of the substitutions W10D+T13N+N101S of SEQ ID NO: 1.

In another aspect, the polypeptide comprises or consists of the substitutions W10D+V166P+Y176W of SEQ ID NO: 1.

In another aspect, the polypeptide comprises or consists of the substitutions W10A+T13N+T14V of SEQ ID NO: 1.

In another aspect, the polypeptide comprises or consists of the substitutions W10R+T14V+N101S of SEQ ID NO: 1.

In another aspect, the polypeptide comprises or consists of the substitutions W10D+N101S+N153S of SEQ ID NO: 1.

In another aspect, the polypeptide comprises or consists of the substitutions W10A+T14V+F37Y of SEQ ID NO: 1.

In another aspect, the polypeptide comprises or consists of the substitutions T13N+T14V+R202E of SEQ ID NO: 1.

In another aspect, the polypeptide polypeptide comprises or consists of the substitutions W10A+T14V+N153S of SEQ ID NO: 1.

In another aspect, the polypeptide comprises or consists of the substitutions T13N+V166P+Y176W of SEQ ID NO: 1.

In another aspect, the polypeptide comprises or consists of the substitutions T14V+V166P+Y176W of SEQ ID NO: 1.

In another aspect, the polypeptide comprises or consists of the substitutions W10A+V166P+Y176W of SEQ ID NO: 1.

In another aspect, the polypeptide comprises or consists of the substitutions W10D+V166P+Y176W of SEQ ID NO: 1.

In another aspect, the polypeptide comprises or consists of the substitutions W10A+T14V+V166P+Y176W of SEQ ID NO: 1.

In another aspect, the polypeptide comprises or consists of the substitutions W10D+N101S+V166P+Y176W of SEQ ID NO: 1.

In another aspect, the polypeptide comprises or consists of the substitutions W10D+T14V+E118R+T122D of SEQ ID NO: 1.

In another aspect, the polypeptide comprises or consists of the substitutions W10D+V166P+Y176W+R202E of SEQ ID NO: 1.

In another aspect, the polypeptide comprises or consists of the substitutions W10A+T13N+E118R+T122D of SEQ ID NO: 1.

In another aspect, the polypeptide comprises or consists of the substitutions F37Y+V166P+Y176W+R202E of SEQ ID NO: 1.

In another aspect, the polypeptide comprises or consists of the substitutions W10A+E118R+T122D+R202E of SEQ ID NO: 1.

In another aspect, the polypeptide comprises or consists of the substitutions D6S+W10D+N101S+V166P of SEQ ID NO: 1.

In another aspect, the polypeptide comprises or consists of the substitutions D6S+G9P+W10D+N101S of the polypeptide of SEQ ID NO: 1.

In another aspect, the polypeptide comprises or consists of the substitutions D6S+W10D+T36E+N101S of SEQ ID NO: 1.

In another aspect, the polypeptide comprises or consists of the substitutions G9P+W10D+N101S+F175N of SEQ ID NO: 1.

In another aspect, the polypeptide comprises or consists of the substitutions G9P+W10D+N101S+S129G of SEQ ID NO: 1.

In another aspect, the polypeptide comprises or consists of the substitutions W10D+T36E+F37E+N101S of SEQ ID NO: 1.

In another aspect, the polypeptide comprises or consists of the substitutions W10D+S40P+N101S+F175N of SEQ ID NO: 1.

In another aspect, the polypeptide comprises or consists of the substitutions W10D+Y20W+N101S+F175N of SEQ ID NO: 1.

In another aspect, the polypeptide comprises or consists of the substitutions 12V+G9P+W10D+N101S of the polypeptide of SEQ ID NO: 1.

In another aspect, the polypeptide comprises or consists of the substitutions W10D+Q63R+A65D+N101S of SEQ ID NO: 1.

In another aspect, the polypeptide comprises or consists of the substitutions W10D+N101S+H125K+W131Y of SEQ ID NO: 1.

In another aspect, the polypeptide comprises or consists of the substitutions T13N+T14V+V166P+Y176W of SEQ ID NO: 1.

In another aspect, the polypeptide comprises or consists of the substitutions W10A+T13N+V166P+Y176W of SEQ ID NO: 1.

In another aspect, the polypeptide comprises or consists of the substitutions F37Y+V166P+Y176W+R202E of SEQ ID NO: 1.

In another aspect, the polypeptide comprises or consists of the substitutions W10A+V166P+Y176W+R202E of SEQ ID NO: 1.

In another aspect, the polypeptide comprises or consists of the substitutions W10A+F37Y+V166P+Y176W of SEQ ID NO: 1.

In another aspect, the polypeptide comprises or consists of the substitutions W10A+E118R+T122D+V166P+Y176W of SEQ ID NO: 1.

In another aspect, the polypeptide comprises or consists of the substitutions W10A+T13N+T14V+V166P+Y176W of SEQ ID NO: 1.

In another aspect, the polypeptide comprises or consists of the substitutions W10A+T13N+T122D+V166P+Y176W of SEQ ID NO: 1.

In another aspect, the polypeptide comprises or consists of the substitutions R1Q+W10A+T13N+T14V+V166P+Y176W of SEQ ID NO: 1.

The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding domain.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Alternatively, the amino acid changes are of such a nature that the physico-chemical properties of the polypeptides are altered. For example, amino acid changes may improve the thermal stability of the polypeptide, alter the substrate specificity, change the pH optimum, and the like.

Essential amino acids in a polypeptide can be identified according to procedures known in the art, such as site-directed mutagenesis or alanine-scanning mutagenesis (Cunningham and Wells, 1989, Science 244:1081-1085). In the latter technique, single alanine mutations are introduced at every residue in the molecule, and the resultant molecules are tested for lysozyme activity to identify amino acid residues that are critical to the activity of the molecule. See also, Hilton et al., 1996, J. Biol. Chem. 271:4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255:306-312; Smith et al., 1992, J. Mol. Biol. 224:899-904; Wlodaver et al., 1992, FEBS Lett. 309:59-64. The identity of essential amino acids can also be inferred from an alignment with a related polypeptide, and/or be inferred from sequence homology and conserved catalytic machinery with a related polypeptide or within a polypeptide or protein family with polypeptides/proteins descending from a common ancestor, typically having similar three-dimensional structures, functions, and significant sequence similarity. Additionally or alternatively, protein structure prediction tools can be used for protein structure modelling to identify essential amino acids and/or active sites of polypeptides. See, for example, Jumper et al., 2021, “Highly accurate protein structure prediction with AlphaFold”, Nature 596:583-589.

The polypeptide may consist of 200 to 250 amino acids, e.g., 210 to 240, 215 to 235, and 220 to 240 amino acids.

In an embodiment, the polypeptide has improved thermostability compared to SEQ ID NO 1 or amino acids 20 to 227 of SEQ ID NO: 2. The improved thermostability is measured of at least 5 °C, at least 5.5 °C, at least 6 °C, at least 6.5 °C, at least 7 °C, at least 7.5 °C, at least 8 °C, at least 8.5 °C, at least 9 °C, at least 9.5 °C, at least 10 °C compared to a polypeptide having SEQ ID NO:1, or amino acids 20 to 227 of SEQ ID NO: 2.

The polypeptide may be a fusion polypeptide comprising a polypeptide of the invention.

In an aspect, the polypeptide is isolated.

In another aspect, the polypeptide is purified.

### Wildtype Lysozymes

A wild type lysozyme is SEQ ID NO 1 or amino acids 20 to 227 of SEQ ID NO: 2 may be obtained from microorganisms of any genus. For purposes of the present invention, the term “obtained from” as used herein in connection with a given source shall mean that the wild type encoded by a polynucleotide is produced by the source or by a strain in which the polynucleotide from the source has been inserted. In one aspect, the wild type is secreted extracellularly.

In another aspect, the wild type lysozyme is isolated from an Acremonium cellulolyticus, Aspergillus aculeatus, Aspergillus awamori, Aspergillus foetidus, Aspergillus fumigatus, Aspergillus japonicus, Aspergillus nidulans, Aspergillus niger, Aspergillus oryzae, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola grisea, Humicola insolens, Humicola lanuginosa, Irpex lacteus, Mucor miehei, Myceliophthora thermophila, Neurospora crassa, Penicillium funiculosum, Penicillium purpurogenum, Phanerochaete chrysosporium, Thielavia achromatica, Thielavia albomyces, Thielavia albopilosa, Thielavia australeinsis, Thielavia fimeti, Thielavia microspora, Thielavia ovispora, Thielavia peruviana, Thielavia setosa, Thielavia spededonium, Thielavia subthermophila, Thielavia terrestris, Trichoderma harzianum, Trichoderma koningii, Trichoderma longibrachiatum, Trichoderma reesei, or Trichoderma viride polypeptide,.

In another aspect, the wild type lysozyme is an Aspergillus aculeatus or an Acremonium alcalophilum polypeptide, e.g., a polypeptide obtained from Aspergillus aculeatus CBS 172.66 or Acremonium alcalophilum CBS 114.92.

It will be understood that for the aforementioned species, the invention encompasses both the perfect and imperfect states, and other taxonomic equivalents, e.g., anamorphs, regardless of the species name by which they are known. Those skilled in the art will readily recognize the identity of appropriate equivalents.

Strains of these species are readily accessible to the public in a number of culture collections, such as the American Type Culture Collection (ATCC), Deutsche Sammlung von Mikroorganismen und Zellkulturen GmbH (DSMZ), Centraalbureau Voor Schimmelcultures (CBS), and Agricultural Research Service Patent Culture Collection, Northern Regional Research Center (NRRL).

The wild type lysozyme may be identified and obtained from other sources including microorganisms isolated from nature (e.g., soil, composts, water, etc.) or DNA samples obtained directly from natural materials (e.g., soil, composts, water, etc.) using the above-mentioned probes. Techniques for isolating microorganisms and DNA directly from natural habitats are well known in the art. A polynucleotide encoding the polypeptide of the wild type lysozyme may then be obtained by similarly screening a genomic DNA or cDNA library of another microorganism or mixed DNA sample. Once a polynucleotide encoding a polypeptide of wild type lysozyme has been detected with the probe(s), the polynucleotide can be isolated or cloned by utilizing techniques that are known to those of ordinary skill in the art (see, e.g., Sambrook et al., 1989, supra).

### Preparation of the Polypeptide of the Invention

The present invention also relates to methods for obtaining a a polypeptide having lysozyme activity, comprising: (a) introducing into a substitution at one or more positions corresponding to positions 1, 2, 6, 9, 10, 13, 14, 20, 36, 37, 40, 41, 63, 65, 101, 103, 118, 122, 125, 126, 129 131, 143, 153, 155, 166 174, 175, 176, 188 and 202 of SEQ ID NO:1; and (b) recovering the polypeptide.

The polypeptide can be prepared using any mutagenesis procedure known in the art, such as site-directed mutagenesis, synthetic gene construction, semi-synthetic gene construction, random mutagenesis, shuffling, etc.

Site-directed mutagenesis is a technique in which one or more mutations are introduced at one or more defined sites in a polynucleotide encoding the parent.

Site-directed mutagenesis can be accomplished in vitro by PCR involving the use of oligonucleotide primers containing the desired mutation. Site-directed mutagenesis can also be performed in vitro by cassette mutagenesis involving the cleavage by a restriction enzyme at a site in the plasmid comprising a polynucleotide encoding the parent and subsequent ligation of an oligonucleotide containing the mutation in the polynucleotide. Usually the restriction enzyme that digests the plasmid and the oligonucleotide is the same, permitting sticky ends of the plasmid and the insert to ligate to one another. See, e.g., Scherer and Davis, 1979, Proc. Natl. Acad. Sci. USA 76:4949-4955; and Barton et al., 1990, Nucleic Acids Res. 18:7349-4966.

Site-directed mutagenesis can also be accomplished in vivo by methods known in the art. See, e.g., US 2004/0171154; Storici et al., 2001, Nature Biotechnol. 19:773-776; Kren et al., 1998, Nat. Med. 4:285-290; and Calissano and Macino, 1996, Fungal Genet. Newslett. 43:15-16.

Any site-directed mutagenesis procedure can be used in the present invention. There are many commercial kits available that can be used to prepare polypeptides of the invention.

Synthetic gene construction entails in vitro synthesis of a designed polynucleotide molecule to encode a polypeptide of interest. Gene synthesis can be performed utilizing a number of techniques, such as the multiplex microchip-based technology described by Tian et al., 2004, Nature 432:1050-1054, and similar technologies wherein oligonucleotides are synthesized and assembled upon photo-programmable microfluidic chips.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241:53-57; Bowie and Sauer, 1989, Proc. Natl. Acad. Sci. USA 86:2152-2156; WO 95/17413; or WO 95/22625. Other methods that can be used include error-prone PCR, phage display (e.g., Lowman et al., 1991, Biochemistry 30:10832-10837; U.S. Pat. No. 5,223,409; WO 92/06204) and region-directed mutagenesis (Derbyshire et al., 1986, Gene 46:145; Ner et al., 1988, DNA 7:127).

Mutagenesis/shuffling methods can be combined with high-throughput, automated screening methods to detect activity of cloned, mutagenized polypeptides expressed by host cells (Ness et al., 1999, Nature Biotechnology 17:893-896). Mutagenized DNA molecules that encode active polypeptides can be recovered from the host cells and rapidly sequenced using standard methods in the art. These methods allow the rapid determination of the importance of individual amino acid residues in a polypeptide.

Semi-synthetic gene construction is accomplished by combining aspects of synthetic gene construction, and/or site-directed mutagenesis, and/or random mutagenesis, and/or shuffling. Semi-synthetic construction is typified by a process utilizing polynucleotide fragments that are synthesized, in combination with PCR techniques. Defined regions of genes may thus be synthesized de novo, while other regions may be amplified using site-specific mutagenic primers, while yet other regions may be subjected to error-prone PCR or non-error prone PCR amplification. Polynucleotide subsequences may then be shuffled.

### Polynucleotides

The present invention also relates to polynucleotides encoding a polypeptide of the present invention.

The polynucleotide may be a genomic DNA, a cDNA, a synthetic DNA, a synthetic RNA, a mRNA, or a combination thereof.

In an aspect, the polynucleotide is isolated.

In another aspect, the polynucleotide is purified.

### Nucleic Acid Constructs

The present invention also relates to nucleic acid constructs comprising a polynucleotide encoding a polypeptide of the present invention operably linked to one or more control sequences that direct the expression of the coding sequence in a suitable host cell under conditions compatible with the control sequences.

The polynucleotide may be manipulated in a variety of ways to provide for expression of a polypeptide. Manipulation of the polynucleotide prior to its insertion into a vector may be desirable or necessary depending on the expression vector. The techniques for modifying polynucleotides utilizing recombinant DNA methods are well known in the art.

### Promoters

The control sequence may be a promoter, a polynucleotide recognized by a host cell for expression of a polynucleotide encoding a polypeptide of the present invention. The promoter contains transcriptional control sequences that mediate the expression of the polypeptide. The promoter may be any polynucleotide that shows transcriptional activity in the host cell including mutant, truncated, and hybrid promoters, and may be obtained from genes encoding extracellular or intracellular polypeptides either homologous or heterologous to the host cell.

Examples of suitable promoters for directing transcription of the polynucleotide of the present invention in a filamentous fungal host cell are promoters obtained from Aspergillus, Fusarium, Rhizomucor and Trichoderma cells, such as the promoters described in Mukherjee et al., 2013, “Trichoderma: Biology and Applications”, and by Schmoll and Dattenbock, 2016, “Gene Expression Systems in Fungi: Advancements and Applications”, Fungal Biology.

### Terminators

The control sequence may also be a transcription terminator, which is recognized by a host cell to terminate transcription. The terminator is operably linked to the 3′-terminus of the polynucleotide encoding the polypeptide. Any terminator that is functional in the host cell may be used in the present invention.

Preferred terminators for filamentous fungal host cells may be obtained from Aspergillus or Trichoderma species, such as obtained from the genes for Aspergillus niger glucoamylase, Trichoderma reesei beta-glucosidase, Trichoderma reesei cellobiohydrolase I, and Trichoderma reesei endoglucanase I, such as the terminators described in Mukherjee et al., 2013, “Trichoderma: Biology and Applications”, and by Schmoll and Dattenbock, 2016, “Gene Expression Systems in Fungi: Advancements and Applications”, Fungal Biology.

mRNA Stabilizers

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryIIIA gene (WO 94/25612) and a Bacillus subtilis SP82 gene (Hue et al., 1995, J. Bacteriol. 177:3465-3471).

Examples of mRNA stabilizer regions for fungal cells are described in Geisberg et al., 2014, Cell 156 (4): 812-824, and in Morozov et al., 2006, Eukaryotic Cell 5 (11): 1838-1846.

### Leader Sequences

The control sequence may also be a leader, a nontranslated region of an mRNA that is important for translation by the host cell. The leader is operably linked to the 5′-terminus of the polynucleotide encoding the polypeptide. Any leader that is functional in the host cell may be used.

Preferred leaders for filamentous fungal host cells may be obtained from the genes for Aspergillus oryzae TAKA amylase and Aspergillus nidulans triose phosphate isomerase.

### Polyadenylation Sequences

The control sequence may also be a polyadenylation sequence, a sequence operably linked to the 3′-terminus of the polynucleotide and, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase, Aspergillus oryzae TAKA amylase, and Fusarium oxysporum trypsin-like protease.

### Signal Peptides

The control sequence may also be a signal peptide coding region that encodes a signal peptide linked to the N-terminus of a polypeptide and directs the polypeptide into the cell's secretory pathway. The 5′-end of the coding sequence of the polynucleotide may inherently contain a signal peptide coding sequence naturally linked in translation reading frame with the segment of the coding sequence that encodes the polypeptide. Alternatively, the 5′-end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the polypeptide. However, any signal peptide coding sequence that directs the expressed polypeptide into the secretory pathway of a host cell may be used.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Humicola lanuginosa lipase, and Rhizomucor miehei aspartic proteinase, such as the signal peptide described by Xu et al., 2018, Biotechnology Letters 40:949-955

### Propeptides

The control sequence may also be a propeptide coding sequence that encodes a propeptide positioned at the N-terminus of a polypeptide. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of a polypeptide and the signal peptide sequence is positioned next to the N-terminus of the propeptide sequence.

### Regulatory Sequences

It may also be desirable to add regulatory sequences that regulate expression of the polypeptide relative to the growth of the host cell. Examples of regulatory sequences are those that cause expression of the gene to be turned on or off in response to a chemical or physical stimulus, including the presence of a regulatory compound. In filamentous fungi, the Aspergillus niger glucoamylase promoter, Aspergillus oryzae TAKA alpha-amylase promoter, and Aspergillus oryzae glucoamylase promoter, Trichoderma reesei cellobiohydrolase I promoter, and Trichoderma reesei cellobiohydrolase II promoter may be used. Other examples of regulatory sequences are those that allow for gene amplification.

### Transcription Factors

The control sequence may also be a transcription factor, a polynucleotide encoding a polynucleotide-specific DNA-binding polypeptide that controls the rate of the transcription of genetic information from DNA to mRNA by binding to a specific polynucleotide sequence. The transcription factor may function alone and/or together with one or more other polypeptides or transcription factors in a complex by promoting or blocking the recruitment of RNA polymerase. Transcription factors are characterized by comprising at least one DNA-binding domain which often attaches to a specific DNA sequence adjacent to the genetic elements which are regulated by the transcription factor. The transcription factor may regulate the expression of a protein of interest either directly, i.e., by activating the transcription of the gene encoding the protein of interest by binding to its promoter, or indirectly, i.e., by activating the transcription of a further transcription factor which regulates the transcription of the gene encoding the protein of interest, such as by binding to the promoter of the further transcription factor. Suitable transcription factors for fungal host cells are described in WO 2017/144177. Suitable transcription factors for prokaryotic host cells are described in Seshasayee et al., 2011, Subcellular Biochemistry 52:7-23, as well in Balleza et al., 2009, FEMS Microbiol. Rev. 33 (1): 133-151.

### Expression Vectors

The present invention also relates to recombinant expression vectors comprising a polynucleotide encoding a polypeptide of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the polypeptide at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

The vector preferably contains at least one element that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous recombination, such as homology-directed repair (HDR), or non-homologous recombination, such as non-homologous end-joining (NHEJ).

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

More than one copy of a polynucleotide of the present invention may be inserted into a host cell to increase production of a polypeptide. For example, 2 or 3 or 4 or 5 or more copies are inserted into a host cell. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

### Host Cells

The present invention also relates to recombinant host cells, comprising a polynucleotide of the present invention operably linked to one or more control sequences that direct the production of a polypeptide of the present invention.

A construct or vector comprising a polynucleotide is introduced into a host cell so that the construct or vector is maintained as a chromosomal integrant or as a self-replicating extra-chromosomal vector as described earlier. The choice of a host cell will to a large extent depend upon the gene encoding the polypeptide and its source. The recombinant host cell may comprise a single copy, or at least two copies, e.g., three, four, five, or more copies of the polynucleotide of the present invention.

The host cell may be any cell useful in the recombinant production of a polypeptide of the invention, e.g., a prokaryotic cell or a fungal cell.

The host cell may be any microbial cell useful in the recombinant production of a polypeptide of the present invention, e.g., a prokaryotic cell or a fungal cell.

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolismis obligately aerobic. In contrast, vegetative growth by yeasts such as Saccharomyces cerevisiae is by budding of a unicellular thallus and carbon catabolismmay be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell. In a preferred embodiment, the filamentous fungal host cell is an Aspergillus, Trichoderma or Fusarium cell. In a further preferred embodiment, the filamentous fungal host cell is an Aspergillus niger, Aspergillus oryzae, Trichoderma reesei, or Fusarium venenatum cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Talaromyces emersonii, Thielavia terrestris, Trametes villosa, Trametes versicolor, Trichoderma harzianum, Trichoderma koningii, Trichoderma longibrachiatum, Trichoderma reesei, or Trichoderma viride cell.

In an aspect, the host cell is isolated.

In another aspect, the host cell is purified.

### Methods of Production

The present invention also relates to methods of producing a polypeptide of the present invention, comprising (a) cultivating a recombinant host cell of the present invention under conditions conducive for production of the polypeptide; and optionally (b) recovering the polypeptide.

The host cell is cultivated in a nutrient medium suitable for production of the polypeptide using methods known in the art. For example, the cells may be cultivated by shake flask cultivation, or small-scale or large-scale fermentation (including continuous, batch, fed-batch, or solid state fermentations) in laboratory or industrial fermentors in a suitable medium and under conditions allowing the polypeptide to be expressed and/or isolated. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The polypeptide may be detected using methods known in the art that are specific for the polypeptide, including, but not limited to, the use of specific antibodies, formation of an enzyme product, disappearance of an enzyme substrate, or an enzyme assay determining the relative or specific activity of the polypeptide.

The polypeptide may be recovered from the medium using methods known in the art, including, but not limited to, collection, centrifugation, filtration, extraction, spray-drying, evaporation, or precipitation. In one aspect, the whole fermentation broth is recovered. In another aspect, a cell-free fermentation broth comprising the polypeptide is recovered.

The polypeptide may be purified by a variety of procedures known in the art to obtain substantially pure polypeptides and/or fragments (see, e.g., Wingfield, 2015, Current Protocols in Protein Science; 80 (1): 6.1.1-6.1.35; Labrou, 2014, Protein Downstream Processing, 1129:3-10).

In an alternative aspect, the polypeptide is not recovered.

### Lysozyme Granules

The present invention also relates to enzyme granules/particles comprising a polypeptide of the invention. In an embodiment, the granule comprises a core, and optionally one or more coatings (outer layers) surrounding the core.

The core may have a diameter, measured as equivalent spherical diameter (volume based average particle size), of 20-2000 μm, particularly 50-1500 μm, 100-1500 μm or 250-1200 μm. The core diameter, measured as equivalent spherical diameter, can be determined using laser diffraction, such as using a Malvern Mastersizer and/or the method described under ISO13320 (2020).

In an embodiment, the core comprises a polypeptide of the present invention.

The core may include additional materials such as fillers, fiber materials (cellulose or synthetic fibers), stabilizing agents, solubilizing agents, suspension agents, viscosity regulating agents, light spheres, plasticizers, salts, lubricants and fragrances.

The core may include a binder, such as synthetic polymer, wax, fat, or carbohydrate.

The core may include a salt of a multivalent cation, a reducing agent, an antioxidant, a peroxide decomposing catalyst and/or an acidic buffer component, typically as a homogenous blend.

The core may include an inert particle with the polypeptide absorbed into it, or applied onto the surface, e.g., by fluid bed coating.

The core may have a diameter of 20-2000 μm, particularly 50-1500 μm, 100-1500 μm or 250-1200 μm.

The core may be surrounded by at least one coating, e.g., to improve the storage stability, to reduce dust formation during handling, or for coloring the granule. The optional coating(s) may include a salt coating, or other suitable coating materials, such as polyethylene glycol (PEG), methyl hydroxy-propyl cellulose (MHPC) and polyvinyl alcohol (PVA).

The coating may be applied in an amount of at least 0.1% by weight of the core, e.g., at least 0.5%, at least 1%, at least 5%, at least 10%, or at least 15%. The amount may be at most 100%, 70%, 50%, 40% or 30%.

The coating is preferably at least 0.1 μm thick, particularly at least 0.5 μm, at least 1 μm or at least 5 μm. In some embodiments, the thickness of the coating is below 100 μm, such as below 60 μm, or below 40 μm.

The coating should encapsulate the core unit by forming a substantially continuous layer. A substantially continuous layer is to be understood as a coating having few or no holes, so that the core unit has few or no uncoated areas. The layer or coating should, in particular, be homogeneous in thickness.

The coating can further contain other materials as known in the art, e.g., fillers, antisticking agents, pigments, dyes, plasticizers and/or binders, such as titanium dioxide, kaolin, calcium carbonate or talc.

A salt coating may comprise at least 60% by weight of a salt, e.g., at least 65%, at least 70%, at least 75%, at least 80%, at least 85%, at least 90%, at least 95% or at least 99% by weight.

To provide acceptable protection, the salt coating is preferably at least 0.1 μm thick, e.g., at least 0.5 μm, at least 1 μm, at least 2 μm, at least 4 μm, at least 5 μm, or at least 8 μm. In a particular embodiment, the thickness of the salt coating is below 100 μm, such as below 60 μm, or below 40 μm.

The salt may be added from a salt solution where the salt is completely dissolved or from a salt suspension wherein the fine particles are less than 50 μm, such as less than 10 μm or less than 5 μm.

The salt coating may comprise a single salt or a mixture of two or more salts. The salt may be water soluble, in particular, having a solubility at least 0.1 g in 100 g of water at 20 °C, preferably at least 0.5 g per 100 g water, e.g., at least 1 g per 100 g water, e.g., at least 5 g per 100 g water.

The salt may be an inorganic salt, e.g., salts of sulfate, sulfite, phosphate, phosphonate, nitrate, chloride or carbonate or salts of simple organic acids (less than 10 carbon atoms, e.g., 6 or less carbon atoms) such as citrate, malonate or acetate. Examples of cations in these salts are alkali or earth alkali metal ions, the ammonium ion or metal ions of the first transition series, such as sodium, potassium, magnesium, calcium, zinc or aluminum. Examples of anions include chloride, bromide, iodide, sulfate, sulfite, bisulfite, thiosulfate, phosphate, monobasic phosphate, dibasic phosphate, hypophosphite, dihydrogen pyrophosphate, tetraborate, borate, carbonate, bicarbonate, metasilicate, citrate, malate, maleate, malonate, succinate, lactate, formate, acetate, butyrate, propionate, benzoate, tartrate, ascorbate or gluconate. In particular, alkali- or earth alkali metal salts of sulfate, sulfite, phosphate, phosphonate, nitrate, chloride or carbonate or salts of simple organic acids such as citrate, malonate or acetate may be used.

The salt in the coating may have a constant humidity at 20 °C above 60%, particularly above 70%, above 80% or above 85%, or it may be another hydrate form of such a salt (e.g., anhydrate). The salt coating may be as described in WO 00/01793 or WO 2006/034710.

Specific examples of suitable salts are NaCl (CH_{20 °C}=76%), Na₂CO₃ (CH_{20 °C}=92%), NaNO₃ (CH_{20 °C}=73%), Na₂HPO₄ (CH_{20 °C}=95%), Na₃PO₄ (CH_{25 °C}=92%), NH₄Cl (CH_{20 °C}=79.5%), (NH₄)₂HPO₄ (CH_{20 °C}=93,0%), NH₄H₂PO₄ (CH_{20 °C}=93.1%), (NH₄)₂SO₄ (CH_{20 °C}=81.1%), KCl (CH_{20 °C}=85%), K₂HPO₄ (CH_{20 °C}=92%), KH₂PO₄ (CH_{20 °C}=96.5%), KNO₃ (CH_{20 °C}=93.5%), Na₂SO₄ (CH_{20 °C}=93%), K₂SO₄ (CH_{20 °C}=98%), KHSO₄ (CH_{20 °C}=86%), MgSO₄ (CH_{20 °C}=90%), ZnSO₄, (CH_{20 °C}=90%) and sodium citrate (CH_{25 °C}=86%). Other examples include NaH₂PO₄, (NH₄)H₂PO₄, CuSO₄, Mg(NO₃)₂ and magnesium acetate.

The salt may be in anhydrous form, or it may be a hydrated salt, i.e., a crystalline salt hydrate with bound water(s) of crystallization, such as described in WO 99/32595. Specific examples include anhydrous sodium sulfate (Na₂SO₄), anhydrous magnesium sulfate (MgSO₄), magnesium sulfate heptahydrate (MgSO₄₇H₂O), zinc sulfate heptahydrate (ZnSO₄·7H₂O), sodium phosphate dibasic heptahydrate (Na₂HPO₄·7H₂O), magnesium nitrate hexahydrate (Mg(NO₃)₂(6H₂O)), sodium citrate dihydrate and magnesium acetate tetrahydrate.

Preferably the salt is applied as a solution of the salt, e.g., using a fluid bed.

The coating materials can be waxy coating materials and film-forming coating materials. Examples of waxy coating materials are poly(ethylene oxide) products (polyethyleneglycol, PEG) with mean molar weights of 1000 to 20000; ethoxylated nonylphenols having from 16 to 50 ethylene oxide units; ethoxylated fatty alcohols in which the alcohol contains from 12 to 20 carbon atoms and in which there are 15 to 80 ethylene oxide units; fatty alcohols; fatty acids; and mono- and di- and triglycerides of fatty acids. Examples of film-forming coating materials suitable for application by fluid bed techniques are given in GB 1483591.

The granule may optionally have one or more additional coatings. Examples of suitable coating materials are polyethylene glycol (PEG), methyl hydroxy-propyl cellulose (MHPC) and polyvinyl alcohol (PVA). Examples of enzyme granules with multiple coatings are described in WO 93/07263 and WO 97/23606.

The core can be prepared by granulating a blend of the ingredients, e.g., by a method comprising granulation techniques such as crystallization, precipitation, pan-coating, fluid bed coating, fluid bed agglomeration, rotary atomization, extrusion, prilling, spheronization, size reduction methods, drum granulation, and/or high shear granulation.

Methods for preparing the core can be found in the Handbook of Powder Technology; Particle size enlargement by C. E. Capes; Vol. 1; 1980; Elsevier. Preparation methods include known feed and granule formulation technologies, e.g.,
(a) Spray dried products, wherein a liquid enzyme-containing solution is atomized in a spray drying tower to form small droplets which during their way down the drying tower dry to form an enzyme-containing particulate material. Very small particles can be produced this way (Michael S. Showell (editor); Powdered detergents; Surfactant Science Series; 1998; Vol. 71; pages 140-142; Marcel Dekker).
(b) Layered products, wherein the enzyme is coated as a layer around a pre-formed inert core particle, wherein an enzyme-containing solution is atomized, typically in a fluid bed apparatus wherein the pre-formed core particles are fluidized, and the enzyme-containing solution adheres to the core particles and dries up to leave a layer of dry enzyme on the surface of the core particle. Particles of a desired size can be obtained this way if a useful core particle of the desired size can be found. This type of product is described in, e.g., WO 97/23606.
(c) Absorbed core particles, wherein rather than coating the polypeptide as a layer around the core, the enzyme is absorbed onto and/or into the surface of the core. Such a process is described in WO 97/39116.
(d) Extrusion or pelletized products, wherein a polypeptide-containing paste is pressed to pellets or under pressure is extruded through a small opening and cut into particles which are subsequently dried. Such particles usually have a considerable size because of the material in which the extrusion opening is made (usually a plate with bore holes) sets a limit on the allowable pressure drop over the extrusion opening. Also, very high extrusion pressures when using a small opening increase heat generation in the enzyme paste, which is harmful to the enzyme (Michael S. Showell (editor); Powdered detergents; Surfactant Science Series; 1998; Vol. 71; pages 140-142; Marcel Dekker).
(e) Prilled products, wherein a polypeptide-containing powder is suspended in molten wax and the suspension is sprayed, e.g., through a rotating disk atomizer, into a cooling chamber where the droplets quickly solidify (Michael S. Showell (editor); Powdered detergents; Surfactant Science Series; 1998; Vol. 71; pages 140-142; Marcel Dekker). The product obtained is one wherein the polypeptide is uniformly distributed throughout an inert material instead of being concentrated on its surface. U.S. Pat. Nos. 4,016,040 and 4,713,245 describe this technique.
(f) Mixer granulation products, wherein a polypeptide-containing liquid is added to a dry powder composition of conventional granulating components. The liquid and the powder in a suitable proportion are mixed and as the moisture of the liquid is absorbed in the dry powder, the components of the dry powder will start to adhere and agglomerate and particles will build up, forming granulates comprising the enzyme. Such a process is described in U.S. Pat. No. 4,106,991, EP 170360, EP 304332, EP 304331, WO 90/09440 and WO 90/09428. In a particular aspect of this process, various high-shear mixers can be used as granulators. Granulates consisting of polypeptide, fillers and binders etc. are mixed with cellulose fibers to reinforce the particles to produce a so-called T-granulate. Reinforced particles, are more robust, and release less enzymatic dust.
(g) Size reduction, wherein the cores are produced by milling or crushing of larger particles, pellets, tablets, briquettes etc. containing the enzyme. The wanted core particle fraction is obtained by sieving the milled or crushed product. Over and undersized particles can be recycled. Size reduction is described in Martin Rhodes (editor); Principles of Powder Technology; 1990; Chapter 10; John Wiley & Sons.
(h) Fluid bed granulation. Fluid bed granulation involves suspending particulates in an air stream and spraying a liquid onto the fluidized particles via nozzles. Particles hit by spray droplets get wetted and become tacky. The tacky particles collide with other particles and adhere to them to form a granule.
(i) The cores may be subjected to drying, such as in a fluid bed drier. Other known methods for drying granules in the feed or enzyme industry can be used by the skilled person. The drying preferably takes place at a product temperature of from 25 to 90 °C. For some enzymes, it is important the cores comprising the polypeptide contain a low amount of water before coating with the salt. If water sensitive enzymes are coated with a salt before excessive water is removed, the excessive water will be trapped within the core and may affect the activity of the enzyme negatively. After drying, the cores preferably contain 0.1-10% w/w water.

Non-dusting granulates may be produced, e.g., as disclosed in U.S. Pat. Nos. 4,106,991 and 4,661,452 and may optionally be coated by methods known in the art.

The granulate may further comprise one or more additional enzymes. Each enzyme will then be present in more granules securing a more uniform distribution of the enzymes, and also reduces the physical segregation of different enzymes due to different particle sizes. Methods for producing multi-enzyme co-granulates is disclosed in the ip.com disclosure IPCOM000200739D. Another example of formulation of enzymes by the use of co-granulates is disclosed in WO 2013/188331.

The present invention also relates to protected enzymes prepared according to the method disclosed in EP 238216.

In an embodiment, the granule further comprises one or more additional enzymes, e.g., hydrolase, isomerase, ligase, lyase, oxidoreductase, and transferase. The one or more additional enzymes are preferably selected from the group consisting of acetylxylan esterase, acylglycerol lipase, amylase, alpha-amylase, beta-amylase, arabinofuranosidase, cellobiohydrolases, cellulase, feruloyl esterase, galactanase, alpha-galactosidase, beta-galactosidase, beta-glucanase, beta-glucosidase, lysophospholipase, lysozyme, alpha-mannosidase, beta-mannosidase (mannanase), phytase, phospholipase A1, phospholipase A2, phospholipase D, protease, pullulanase, pectin esterase, triacylglycerol lipase, xylanase, beta-xylosidase or any combination thereof.

### Liquid Formulations

The present invention also relates to liquid compositions comprising a polypeptide of the invention. The composition may comprise an enzyme stabilizer (examples of which include polyols such as propylene glycol or glycerol, sugar or sugar alcohol, lactic acid, reversible protease inhibitor, boric acid, or a boric acid derivative, e.g., an aromatic borate ester, or a phenyl boronic acid derivative such as 4-formylphenyl boronic acid).

In some embodiments, filler(s) or carrier material(s) are included to increase the volume of such compositions. Suitable filler or carrier materials include, but are not limited to, various salts of sulfate, carbonate and silicate as well as talc, clay and the like. Suitable filler or carrier materials for liquid compositions include, but are not limited to, water or low molecular weight primary and secondary alcohols including polyols and diols. Examples of such alcohols include, but are not limited to, methanol, ethanol, propanol and isopropanol. In some embodiments, the compositions contain from about 5% to about 90% of such materials.

In an aspect, the liquid formulation comprises 20-80% w/w of polyol. In one embodiment, the liquid formulation comprises 0.001-2% w/w preservative.

In another embodiment, the invention relates to liquid formulations comprising:
(A) 0.001-25% w/w of a polypeptide of the present invention;
(B) 20-80% w/w of polyol;
(C) optionally 0.001-2% w/w preservative; and
(D) water.

In another embodiment, the invention relates to liquid formulations comprising:
(A) 0.001-25% w/w of a polypeptide of the present invention;
(B) 0.001-2% w/w preservative;
(C) optionally 20-80% w/w of polyol; and
(D) water.

In another embodiment, the liquid formulation comprises one or more formulating agents, such as a formulating agent selected from the group consisting of polyol, sodium chloride, sodium benzoate, potassium sorbate, sodium sulfate, potassium sulfate, magnesium sulfate, sodium thiosulfate, calcium carbonate, sodium citrate, dextrin, glucose, sucrose, sorbitol, lactose, starch, PVA, acetate and phosphate, preferably selected from the group consisting of sodium sulfate, dextrin, cellulose, sodium thiosulfate, kaolin and calcium carbonate. In one embodiment, the polyols is selected from the group consisting of glycerol, sorbitol, propylene glycol (MPG), ethylene glycol, diethylene glycol, triethylene glycol, 1,2-propylene glycol or 1,3-propylene glycol, dipropylene glycol, polyethylene glycol (PEG) having an average molecular weight below about 600 and polypropylene glycol (PPG) having an average molecular weight below about 600, more preferably selected from the group consisting of glycerol, sorbitol and propylene glycol (MPG) or any combination thereof.

In another embodiment, the liquid formulation comprises 20-80% polyol (i.e., total amount of polyol), e.g., 25-75% polyol, 30-70% polyol, 35-65% polyol, or 40-60% polyol. In one embodiment, the liquid formulation comprises 20-80% polyol, e.g., 25-75% polyol, 30-70% polyol, 35-65% polyol, or 40-60% polyol, wherein the polyol is selected from the group consisting of glycerol, sorbitol, propylene glycol (MPG), ethylene glycol, diethylene glycol, triethylene glycol, 1,2-propylene glycol or 1,3-propylene glycol, dipropylene glycol, polyethylene glycol (PEG) having an average molecular weight below about 600 and polypropylene glycol (PPG) having an average molecular weight below about 600. In one embodiment, the liquid formulation comprises 20-80% polyol (i.e., total amount of polyol), e.g., 25-75% polyol, 30-70% polyol, 35-65% polyol, or 40-60% polyol, wherein the polyol is selected from the group consisting of glycerol, sorbitol and propylene glycol (MPG).

In another embodiment, the preservative is selected from the group consisting of sodium sorbate, potassium sorbate, sodium benzoate and potassium benzoate or any combination thereof. In one embodiment, the liquid formulation comprises 0.02-1.5% w/w preservative, e.g., 0.05-1% w/w preservative or 0.1-0.5% w/w preservative. In one embodiment, the liquid formulation comprises 0.001-2% w/w preservative (i.e., total amount of preservative), e.g., 0.02-1.5% w/w preservative, 0.05-1% w/w preservative, or 0.1-0.5% w/w preservative, wherein the preservative is selected from the group consisting of sodium sorbate, potassium sorbate, sodium benzoate and potassium benzoate or any combination thereof.

In another embodiment, the liquid formulation further comprises one or more additional enzymes, e.g., hydrolase, isomerase, ligase, lyase, oxidoreductase, and transferase. The one or more additional enzymes are preferably selected from the group consisting of acetylxylan esterase, acylglycerol lipase, amylase, alpha-amylase, beta-amylase, arabinofuranosidase, cellobiohydrolases, cellulase, feruloyl esterase, galactanase, alpha-galactosidase, beta-galactosidase, beta-glucanase, beta-glucosidase, lysophospholipase, lysozyme, alpha-mannosidase, beta-mannosidase (mannanase), phytase, phospholipase A1, phospholipase A2, phospholipase D, protease, pullulanase, pectin esterase, triacylglycerol lipase, xylanase, beta-xylosidase or any combination thereof.

### Compositions

In a still further aspect, the present invention relates to compositions comprising a polypeptide of the present invention having antimicrobial and/or lysozyme activity.

The composition may comprise a polypeptide of the invention as the major enzymatic component, e.g., a mono-component composition.

The compositions may be prepared in accordance with methods known in the art and may be in the form of a liquid or a dry composition. For instance, the polypeptide composition may be in the form of a granulate or a microgranulate. The polypeptide to be included in the composition may be stabilized in accordance with methods known in the art.

Examples are given below of preferred uses of the polypeptide compositions of the invention. The dosage of the polypeptide composition of the invention and other conditions under which the composition is used may be determined on the basis of methods known in the art.

### Animal Feed and Animal Feed Additive

A lysozyme of the invention is used in animal feed. In an embodiment, the present invention provides a method for preparing an animal feed composition comprising adding a lysozyme of the present invention to one or more animal feed ingredients.

The invention relates to polypeptides which have improved lysozyme activity against the peptidoglycans found in the cell walls of undesired intestinal microbiota, thereby rendering them suitable for use in animal feed to improve the animal health. The lysozymes of the invention have improved activity compared to the lysozyme described in WO2013/076253. A large subset of the GH25 lysozymes have surprisingly been found to furthermore have lysozyme activity against Lactobacillus johnsonii. Lactobacillus johnsonii is an important bacterium of the intestinal flora of animals. Without being bound to a particular theory, it is believed that removal of dead Lactobacillus johnsonii cells from the intestinal flora, by means of enzymatic lyses of the partially degraded bacterial cell wall, to be an important contributor to intestinal health of an animal.

An aspect of the invention is directed to a method of improving the intestinal health in an animal comprising reducing the amount of dead cells, or cell wall debris, in the digestive tract of said animal, comprising feeding the animal with a feed or feed additive comprising a polypeptide as defined by the invention.

A lysozyme of the present invention may for example be used to stabilize the healthy microflora of animals, in particular livestock such as, but not limited to, sheep, goats, cattle (including, but not limited to, beef cattle, cows, and young calves), deer, pigs or swine (including, but not limited to, piglets, growing pigs, and sows), poultry (including, but not limited to, geese, turkeys, ducks and chicken such as broilers, chicks and layers); horses, moose and rabbits but also in fish (including but not limited to salmon, trout, tilapia, catfish and carps; and crustaceans (including but not limited to shrimps and prawns)) by suppressing growth/intestinal colonization of viral, bacterial or parasitic pathogens such as Clostridium perfringens, Escherichia coli and Salmonella, such as Salmonella enterica, Salmonella Typhimurium and Salmonella Mbandaka. In a preferred embodiment, the lysozyme replaces antibiotics in animal diets. In a preferred embodiment a lysozyme is applied to chicken and has anti-microbal activity against Clostridium perfringens.

In a further embodiment a lysozyme of the present invention is used as a feed additive, where it may provide a positive effect on the microbial balance of the chicken digestive tract and in this way improve animal performance.

A lysozyme of the present invention may also be used in animal feed as feed enhancing enzymes that improve feed digestibility to increase the efficiency of its utilization according to WO 00/21381 and WO 04/026334.

The present invention is also directed to methods for using the polypeptides of the present invention having lysozyme activity in animal feed, as well as to feed compositions and feed additives comprising the lysozymes of the invention.

A polypeptide of the present invention may for example be used to stabilize the healthy microflora of animals, in particular livestock such as, but not limited to, sheep, goats, cattle (including, but not limited to, beef cattle, cows, and young calves), deer, pigs or swine (including, but not limited to, piglets, growing pigs, and sows), poultry (including, but not limited to, geese, turkeys, ducks and chicken such as broilers, chicks and layers); horses, moose and rabbits but also in fish (including but not limited to salmon, trout, tilapia, catfish and carps; and crustaceans (including but not limited to shrimps and prawns)) by suppressing growth/intestinal colonization of viral (such as Coronaviridae, Porcine reproductive and respiratory syndrome virus (PRRSV), Persivirus coursing Bovin virus diarre and likewise), parasitic pathogens (coccidian protozoa, Eimeria maxima, Eimeria mitis) or bacterial pathogens such as Clostridium perfringens, Escherichia coli, Campylobacter coli, C. hyointestinalis and C. jejuni, Yersinia ssp., Treponema suis, Brachyspira hyodysenteriae, Lawsonia intracellularis and Salmonella, such as Salmonella enterica, Salmonella Typhimurium and Salmonella Mbandaka.

An aspect of the invention is directed to the use of the polypeptide of the invention:
V. in animal feed;
vi. in animal feed additives;
vii. in the preparation of a composition for use in animal feed; and/or
viii. for improving the intestinal health in an animal.

A further aspect of the invention is directed zootechnical additive for use in feed for poultry or swine, said additive comprising the polypeptide as defined herein.

In the use according to the invention the lysozymes can be fed to the animal before, after, or simultaneously with the diet. The latter is preferred.

In a particular embodiment, the lysozyme, in the form in which it is added to the feed, or when being included in a feed additive, is well defined. Well-defined means that the lysozyme preparation is at least 50% pure as determined by Size-exclusion chromatography (see Example 12 of WO 01/58275). In other particular embodiments the lysozyme preparation is at least 60, 70, 80, 85, 88, 90, 92, 94, or at least 95% pure as determined by this method.

A well-defined lysozyme preparation is advantageous. For instance, it is much easier to dose correctly to the feed a lysozyme that is essentially free from interfering or contaminating other lysozymes. The term dose correctly refers in particular to the objective of obtaining consistent and constant results, and the capability of optimizing dosage based upon the desired effect.

For the use in animal feed, however, the lysozyme need not be pure; it may e.g. include other enzymes, in which case it could be termed a lysozyme preparation.

The lysozyme preparation can be (a) added directly to the feed, or (b) it can be used in the production of one or more intermediate compositions such as feed additives or premixes that is subsequently added to the feed (or used in a treatment process). The degree of purity described above refers to the purity of the original lysozyme preparation, whether used according to (a) or (b) above.

The lysozyme of the present invention could also be used in the prevention of necrotic enteritis and/or Clostridium perfringens.

In an embodiment, the present invention also relates to a method of improving the performance of an animal comprising administering to the animal the animal feed or the animal feed additive of the invention.

The term feed or feed composition means any compound, preparation, mixture, or composition suitable for, or intended for intake by an animal. In the use according to the invention the lysozyme can be fed to the animal before, after, or simultaneously with the diet. The latter is preferred. Such lysozyme compositions may of course be mixed with other enzymes.

The lysozyme can be added to the feed in any form, be it as a relatively pure lysozyme or in admixture with other components intended for addition to animal feed, i.e. in the form of animal feed additives, such as the so-called pre-mixes for animal feed. In a further aspect the present invention relates to compositions for use in animal feed, such as animal feed, and animal feed additives, e.g. premixes.

Examples of preferred uses of the lysozyme or compositions thereof of the present invention are given below. The dosage of the lysozyme and other conditions under which the lysozyme is used may be determined on the basis of methods known in the art.

Apart from the lysozyme of the invention, the animal feed additives of the invention contain at least one fat soluble vitamin, and/or at least one water soluble vitamin, and/or at least one trace mineral, and/or at least one macro mineral.

Further, optional, feed-additive ingredients are colouring agents, e.g. carotenoids such as beta-carotene, astaxanthin, and lutein; stabilisers; growth improving additives and aroma compounds/flavorings, e.g. creosol, anethol, deca-, undeca-and/or dodeca-lactones, ionones, irone, gingerol, piperidine, propylidene phatalide, butylidene phatalide, capsaicin and/or tannin; polyunsaturated fatty acids (PUFAs); reactive oxygen generating species; also, a support may be used that may contain, for example, 40-50% by weight of wood fibres, 8-10% by weight of stearine, 4-5% by weight of curcuma powder, 4-58% by weight of rosemary powder, 22-28% by weight of limestone, 1-3% by weight of a gum, such as gum arabic, 5-50% by weight of sugar and/or starch and 5-15% by weight of water.

A feed or a feed additive of the invention may also comprise at least one other enzyme selected from amongst phytase (EC 3.1.3.8 or 3.1.3.26); xylanase (EC 3.2.1.8); galactanase (EC 3.2.1.89); alpha-galactosidase (EC 3.2.1.22); protease (EC 3.4), phospholipase A1 (EC 3.1.1.32); phospholipase A2 (EC 3.1.1.4); lysophospholipase (EC 3.1.1.5); phospholipase C (3.1.4.3); phospholipase D (EC 3.1.4.4); amylase such as, for example, alpha-amylase (EC 3.2.1.1); and/or beta-glucanase (EC 3.2.1.4 or EC 3.2.1.6).

Examples of polyunsaturated fatty acids are C18, C20 and C22 polyunsaturated fatty acids, such as arachidonic acid, docosahexaenoic acid, eicosapentaenoic acid and gamma-linoleic acid.

Examples of reactive oxygen generating species are chemicals such as perborate, persulphate, or percarbonate; and enzymes such as an oxidase, an oxygenase or a synthetase.

Usually, fat soluble and water-soluble vitamins, as well as trace minerals form part of a so-called premix intended for addition to the feed, whereas macro minerals are usually separately added to the feed. Either of these composition types, when enriched with a protease of the invention, is an animal feed additive of the invention.

In a particular embodiment, the animal feed additive of the invention is intended for being included (or prescribed as having to be included) in animal diets or feed at levels of 0.001 to 1.0%; more particularly 0.005 to 0.5%; or 0.02 to 0.1% (% meaning g additive per 100 g feed). This is so in particular for premixes.

The animal feed typically comprises plant based material. In an embodiment, the plant based material is selected from the group consisting of legumes, cereals, oats, rye, barley, wheat, maize, corn, sorghum, switchgrass, millet, pearl millet, foxtail millet, soybean, wild soybean, beans, lupin, tepary bean, scarlet runner bean, slimjim bean, lima bean, French bean, Broad bean (fava bean), chickpea, lentil, peanut, Spanish peanut, canola, rapeseed (oilseed rape), rice, beet, cabbage, sugar beet, spinach, quinoa, or pea, in a processed form thereof (such as soybean meal, rapeseed meal) or any combination thereof.

The animal feed composition of the invention may contain at least one vegetable protein, such as that derived from or originating from a vegetable, including modified proteins and protein-derivatives. Vegetable proteins may be derived from vegetable protein sources, such as legumes and cereals, for example materials from plants of the families Fabaceae (Leguminosae), Cruciferaceae, Chenopodiaceae, and Poaceae, such as soybean meal, lupin meal and rapeseed meal, Alternatively, the vegetable protein source is material from one or more plants of the family Chenopodiaceae, e.g. beet, sugar beet, spinach or quinoa. Other examples of vegetable protein sources are rapeseed, sunflower seed, cotton seed, and cabbage and cereals such as barley, wheat, rye, oat, maize (corn), rice, triticale, and sorghum.

The animal feed composition of the invention may also contain animal protein, such as Meat and Bone Meal, Feather meal, and/or Fish Meal, typically in an amount of 0-25%. The animal feed composition of the invention may also comprise Dried Destillers Grains with Solubles (DDGS), typically in amounts of 0-30%.

In still further particular embodiments, the animal feed composition of the invention contains 0-80% maize; and/or 0-80% sorghum; and/or 0-70% wheat; and/or 0-70% Barley; and/or 0-30% oats; and/or 0-40% soybean meal; and/or 0-25% fish meal; and/or 0-25% meat and bone meal; and/or 0-20% whey.

Animal diets can e.g., be manufactured as mash feed (non-pelleted) or pelleted feed. Typically, the milled feedstuffs are mixed, and sufficient amounts of essential vitamins and minerals are added according to the specifications for the species in question. Enzymes can be added as solid or liquid enzyme formulations. For example, for mash feed a solid or liquid enzyme formulation may be added before or during the ingredient mixing step. For pelleted feed the (liquid or solid) lysozyme/enzyme preparation may also be added before or during the feed ingredient step. Typically, a liquid lysozyme/enzyme preparation is added after the pelleting step. The enzyme may also be incorporated in a feed additive or premix.

The final enzyme concentration in the diet is within the range of 0.01-200 mg enzyme protein per kg diet, for example in the range of 0.5-25 mg enzyme protein per kg animal diet.

The present invention is further described by the following examples that should not be construed as limiting the scope of the invention.

### Preferred Embodiments of the Invention are Further Defined by the Following Paragraphs:

1. A polypeptide having lysozyme activity comprising a substitution at one or more positions corresponding to position 1, 2, 6, 9, 10, 13, 14, 20, 36, 37, 40, 41, 63, 65, 101, 103, 118, 122, 125, 126, 129 131, 143, 153, 155, 166 174, 175, 176, 188 and 202 of SEQ ID NO: 1, and wherein the polypeptide has at least 60%, at least 65%, at least 70%, at least 75%, at least 80%, at least 85%, at least 90%, at least 95%, at least 96%, at least 97%, at least 98%, or at least 99%, but less than 100% sequence identity sequence identity to SEQ ID NO: 1 or amino acids 20 to 227 of SEQ ID NO: 2.
2. The polypeptide according to paragraph 1, wherein the polypeptide has improved thermostability compared to a polypeptide having sequence of SEQ ID NO:1, or amino acids 20 to 227 of SEQ ID NO: 2.
3. The polypeptide according to paragraphs 1 or 2, wherein the improved thermostability is measured of at least 5 °C, at least 5.5 °C, at least 6 °C, at least 6.5 °C, at least 7 °C, at least 7.5 °C, at least 8 °C, at least 8.5 °C, at least 9 °C, at least 9.5 °C, at least 10 °C compared to a polypeptide having SEQ ID NO:1, or amino acids 20 to 227 of SEQ ID NO: 2.
4. The polypeptide of any one of paragraphs 1-3, wherein the polypeptide comprises 1-20 alterations, e.g., 1-10, such as 1, 2, 3, 4, 5, 6, 7, 8, 9 or 10 alterations.
5. The polypeptide according to any one of the preceding paragraphs, wherein the number of substitutions, e.g., 1-10, such as 1, 2, 3, 4, 5, 6, 7, 8, 9 or 10 substitutions.
6. The polypeptide according to any one of the preceding paragraphs, which comprises a substitution of the amino acid residue at position 1 of SEQ ID NO:1 with Ala, Asn, Asp, Cys, Gln, Glu, Gly, His, Ile, Leu, Lys, Met, Phe, Pro, Ser, Thr, Trp, Tyr, or Val, preferably with Thr or Gln.
7. The polypeptide according to any one of the preceding paragraphs, which comprises a substitution of the amino acid residue at position 10 of SEQ ID NO: 1 with Ala, Arg, Asn, Asp, Cys, Gln, Glu, Gly, His, Ile, Leu, Lys, Met, Phe, Pro, Ser, Thr, Tyr, or Val, preferably with Ala, Asp or Arg.
8. The polypeptide according to any one of the preceding paragraphs, which comprises a substitution of the amino acid residue at position 14 of SEQ ID NO: 1 with Ala, Arg, Asn, Asp, Cys, Gln, Glu, Gly, His, Ile, Leu, Lys, Met, Phe, Pro, Ser, Trp, Tyr, or Val, preferably with Val.
9. The polypeptide according to any one of the preceding paragraphs, which comprises a substitution of the amino acid residue at position 37 of SEQ ID NO: 1 with Ala, Arg, Asn, Asp, Cys, Gln, Glu, Gly, His, Ile, Leu, Lys, Met, Pro, Ser, Thr, Trp, Tyr, or Val, preferably with Asn or Tyr.
10. The polypeptide according to any one of the preceding paragraphs, which comprises a substitution of the amino acid residue at position 41 of SEQ ID NO:1 with Arg, Asn, Asp, Cys, Gln, Glu, Gly, His, Ile, Leu, Lys, Met, Phe, Pro, Ser, Thr, Trp, Tyr, or Val, preferably with Tyr.
11. The polypeptide according to any one of the preceding paragraphs, which comprises a substitution of the amino acid residue at position 103 of SEQ ID NO:1 with Arg, Asn, Asp, Cys, Gln, Glu, Gly, His, Ile, Leu, Lys, Met, Phe, Pro, Ser, Thr, Trp, Tyr, or Val, preferably with Ser.
12. The polypeptide according to any one of the preceding paragraphs, which comprises a substitution of the amino acid residue at position 118 of SEQ ID NO:1 with Ala, Arg, Asn, Asp, Cys, Gln, Gly, His, Ile, Leu, Lys, Met, Phe, Pro, Ser, Thr, Trp, Tyr, or Val, preferably with Arg.
13. The polypeptide according to any one of the preceding paragraphs, which comprises a substitution of the amino acid residue at position 122 of SEQ ID NO:1 with Ala, Arg, Asn, Asp, Cys, Gln, Glu, Gly, His, Ile, Leu, Lys, Met, Phe, Pro, Ser, Trp, Tyr, or Val, preferably with Pro.
14. The polypeptide according to any one of the preceding paragraphs, which comprises a substitution of the amino acid residue at position 125 of SEQ ID NO:1 with Ala, Arg, Asn, Asp, Cys, Gln, Glu, Gly, Ile, Leu, Lys, Met, Phe, Pro, Ser, Thr, Trp, Tyr, or Val, preferably with Pro.
15. The polypeptide according to any one of the preceding paragraphs, which comprises a substitution of the amino acid residue at position 126 of SEQ ID NO:1 with Ala, Arg, Asn, Asp, Cys, Gln, Glu, His, Ile, Leu, Lys, Met, Phe, Pro, Ser, Thr, Trp, Tyr, or Val, preferably with Asn or Pro.
16. The polypeptide according to any one of the preceding paragraphs, which comprises a substitution of the amino acid residue at position 131 of SEQ ID NO:1 with Ala, Arg, Asn, Asp, Cys, Gln, Glu, Gly, His, Ile, Leu, Lys, Met, Phe, Pro, Ser, Thr, Trp or Tyr, preferably with Cys.
17. The polypeptide according to any one of the preceding paragraphs, which comprises a substitution of the amino acid residue at position 143 of SEQ ID NO:1 with Ala, Arg, Asn, Asp, Cys, Glu, Gly, His, Ile, Leu, Lys, Met, Phe, Pro, Ser, Thr, Trp, Tyr, or Val, preferably with Asn.
18. The polypeptide according to any one of the preceding paragraphs, which comprises a substitution of the amino acid residue at position 153 of SEQ ID NO:1 with Ala, Arg, Asp, Cys, Gln, Glu, Gly, His, Ile, Leu, Lys, Met, Phe, Pro, Ser, Thr, Trp, Tyr, or Val, preferably with Ser.
19. The polypeptide according to any one of the preceding paragraphs, which comprises a substitution of the amino acid residue at position 155 of SEQ ID NO:1 with Ala, Arg, Asn, Asp, Gln, Glu, Gly, His, Ile, Leu, Lys, Met, Phe, Pro, Ser, Thr, Trp, Tyr, or Val, preferably with Trp.
20. The polypeptide according to any one of the preceding paragraphs, which comprises a substitution of the amino acid residue at position 174 of SEQ ID NO:1 with Ala, Arg, Asn, Asp, Cys, Gln, Glu, His, Ile, Leu, Lys, Met, Phe, Pro, Ser, Thr, Trp, Tyr, or Val, preferably with Pro.
21. The polypeptide according to any one of the preceding paragraphs, which comprises a substitution of the amino acid residue at position 175 of SEQ ID NO:1 with Ala, Arg, Asn, Asp, Cys, Gln, Glu, Gly, His, Ile, Leu, Lys, Met, Pro, Ser, Thr, Trp, Tyr, or Val, preferably with Ser.
22. The polypeptide according to any one of the preceding paragraphs, which comprises a substitution of the amino acid residue at position 188 of SEQ ID NO:1 with Ala, Arg, Asn, Asp, Cys, Gln, Glu, His, Ile, Leu, Lys, Met, Phe, Pro, Ser, Thr, Trp, Tyr, or Val, preferably with Pro.
23. The polypeptide according to any one of the preceding paragraphs, which comprises a substitution of the amino acid residue at position 202 of SEQ ID NO:1 with Ala, Asn, Asp, Cys, Gln, Glu, Gly, His, Ile, Leu, Lys, Met, Phe, Pro, Ser, Thr, Trp, Tyr, or Val, preferably with Glu.
24. The polypeptide according to any one of the preceding paragraphs, wherein the polypeptide consists of 200 to 250 amino acids, e.g., 210 to 240, 215 to 235, and 220 to 240 amino acids.
25. The polypeptide according to any one of the preceding paragraphs, which comprises substitutions at at least two positions corresponding to any of positions 10, 13, 14, 143, 166, and 176 of SEQ ID NO:1.
26. The polypeptide according to any one of the preceding paragraphs, which comprises substitutions at at least three positions corresponding to any of positions 10, 13, 14, 37, 101, 153, 166, 176, 202 of SEQ ID NO:1.
27. The polypeptide according to any one of the preceding paragraphs, which comprises substitutions at at least four positions corresponding to any of positions 2, 6, 9, 10, 13, 14, 20, 36, 37, 40, 63, 65, 101, 118, 122, 125, 126 129, 131, 166, 175, 176 and 202 of SEQ ID NO:1.
28. The polypeptide according to any one of the preceding paragraphs, which comprises substitutions at at least five positions corresponding to any of positions 1, 10, 13, 14, 118, 122, 166, 176 of SEQ ID NO:1.
29. The polypeptide according to any one of the preceding paragraphs, which comprises a substitution at position corresponding to position 1 of SEQ ID NO: 1, preferably R1Q.
30. The polypeptide according to any one of the preceding paragraphs, which comprises substitutions at positions corresponding to positions 166 and 176 of SEQ ID NO: 1, preferably V166P+Y176W.
31. The polypeptide according to any one of the preceding paragraphs, which comprises substitutions at positions corresponding to positions 10, 166 and 176 of SEQ ID NO: 1, preferably W10A+V166P+Y176W.
32. The polypeptide according to any one of the preceding paragraphs, which comprises substitutions at positions corresponding to positions 14, 166 and 176 of SEQ ID NO: 1, preferably T14V+V166P+Y176W.
33. The polypeptide according to any one of the preceding paragraphs, which comprises substitutions at positions corresponding to positions 10, 14, 166 and 176 of SEQ ID NO: 1 preferably W10A+T14V+V166P+Y176W.
34. The polypeptide according to any one of the preceding paragraphs, which comprises substitutions at positions corresponding to positions 10, 13, 14, 166, 176 of SEQ ID NO: 1, preferably W10A+T13N+T14V+V166P+Y176W.
35. The polypeptide according to any one of the preceding paragraphs, which comprises substitutions at positions corresponding to positions 1, 10, 13, 14, 166 and 176 of SEQ ID NO: 1, preferably R1Q+W10A+T13N+T14V+V166P+Y176W.
36. The polypeptide according to any one of the preceding paragraphs, wherein the polypeptide comprises at least one of the following substitutions or combinations of substitutions:
R1T, R1Q, W10A, W10D, W10R, T14V, F37N, F37Y, A41Y, A103S, E118R, T122P, H₁₂₅P, G126N, G126P, V131C, Q143N, N153S, C155W, G174P, F175S, G188P, R202E, V166P+Y176W,
T14V+N101S+R202E,
W10R+T13N+F37Y,
T13N+T14V+F37Y,
W10D+T13N+N101S,
W10D+N101S+V166P+Y176W,
W10D+V166P+Y176W,
W10A+T13N+T14V,
W10A+E118R+T122D+V166P+Y176W,
W10D+T14V+E118R+T122D,
W10D+V166P+Y176W+R202E,
W10R+T14V+N101S,
W10A+T13N+E118R+T122D,
W10D+N101S+N153S,
W10A+T14V+F37Y,
F37Y+V166P+Y176W+R202E,
W10A+E118R+T122D+R202E,
T13N+T14V+R202E,
W10A+T14V+N153S,
D6S+W10D+N101S+V166P
D6S+G9P+W10D+N101S,
D6S+W10D+T36E+N101S,
G9P+W10D+N101S+F175N,
G9P+W10D+N101S+S129G,
W10D+T36E+F37E+N101S,
W10D+Q143E,
W10D+S40P+N101S+F175N,
W10D+Y20W+N101S+F175N,
12V+G9P+W10D+N101S,
W10D+Q63R+A65D+N101S,
W10D+N101S+H₁₂₅K+W131Y,
T13N+Y176W,
W10A+T14V,
W10D+T14V,
W10R+Y176W,
W10R+V166P,
T13N+V166P+Y176W,
T13N+T14V+V166P+Y176W,
W10A+T13N+V166P+Y176W,
T14V+V166P+Y176W,
W10A+V166P+Y176W,
W10A+T14V+V166P+Y176W
W10D+V166P+Y176W,
W10A+T13N+T14V+V166P+Y176W,
F37Y+V166P+Y176W+R202E,
W10A+T13N+T122D+V166P+Y176W,
W10A+V166P+Y176W+R202E,
W10A+F37Y+V166P+Y176W, and
R1Q+W10A+T13N+T14V+V166P+Y176W
of SEQ ID NO: 1 and, wherein the polypeptide has lysozyme activity and wherein the polypeptide has at least 60%, at least 65%, at least 70%, at least 75%, at least 80%, about 85%, about 90%, about 91%, about 92%, about 93%, about 94%, about 95%, about 96%, about 97%, about 98%, about 99%, but less than 100% sequence identity to the polypeptide of SEQ ID NO: 1 or amino acids 20 to 227 of SEQ ID NO: 2.
37. The polypeptide according to any one of the preceding paragraphs, wherein the polypeptide comprises one or more of the following substitutions at a position corresponding to positions R1Q, W10A, T13N, T14V, V166P, Y176W of SEQ ID NO: 1.
38. A fusion polypeptide comprising the polypeptide according to any one of the preceding paragraphs 1-37 and a second polypeptide.
39. A granule, which comprises:
(a) a core comprising the polypeptide of any one of paragraphs 1-37, and, optionally
(b) a coating consisting of one or more layer(s) surrounding the core.
40. A granule, which comprises:
(c) a core, and
(d) a coating consisting of one or more layer(s) surrounding the core, wherein the coating comprises the variant of any one of paragraphs 1-37.
41. A liquid composition comprising the polypeptide of any one of paragraphs 1-37 and an enzyme stabilizer, e.g., a polyol such as propylene glycol or glycerol, sugar or sugar alcohol, lactic acid, reversible protease inhibitor, boric acid, or a boric acid derivative, e.g., an aromatic borate ester, or a phenyl boronic acid derivative such as 4-formylphenyl boronic acid).
42. The liquid composition of paragraph 41, further comprising a filler or carrier material.
43. The liquid composition of paragraph 41 or 42, further comprising a preservative.
44. A composition comprising the polypeptide of any one of paragraphs 1-37, the granule of paragraphs 39 or 40, or the liquid compositions of any one of paragraphs 35-37.
45. A polynucleotide encoding the polypeptide of any one of paragraphs 1-37, wherein the polynucleotide has at least 80%, at least 85%, at least 90%, at least 95%, at least 96%, at least 97%, at least 98%, or at least 99%, but less than 100% sequence identity sequence identity to SEQ ID NO: 3.
46. The polynucleotide of paragraph 45, which is isolated.
47. The polynucleotide of paragraph 45 or 46, which is purified.
48. A nucleic acid construct or expression vector comprising the polynucleotide of any one of paragraphs 45-47.
49. A recombinant host cell transformed with the polynucleotide of any one of paragraphs 45-47.
50. The recombinant host cell of paragraph 49, which comprises at least two copies, e.g., three, four, or five, or more copies of the polynucleotide of any one of paragraphs 45-47.
51. The recombinant host cell of paragraph 49 or 50, which is a filamentous fungal recombinant host cell, e.g., an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell, in particular, an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Talaromyces emersonii, Thielavia terrestris, Trametes villosa, Trametes versicolor, Trichoderma harzianum, Trichoderma koningii, Trichoderma longibrachiatum, Trichoderma reesei, or Trichoderma viride cell.
52. The recombinant host cell of any one of paragraphs 49-51, which is isolated.
53. The recombinant host cell of any one of paragraphs 49-51, which is purified.
54. A method of producing a polypeptide, comprising:
a) cultivating the host cell of any one of paragraphs 49-53 under conditions suitable for expression of the polypeptide; and
b) recovering the polypeptide.
55. A method for obtaining a polypeptide, comprising introducing a substitution at one or more positions corresponding to positions 1, 2, 6, 9, 10, 13, 14, 20, 36, 37, 40, 41, 63, 65, 101, 103, 118, 122, 125, 126, 129 131, 143, 153, 155, 166 174, 175, 176, 188 and 202 SEQ ID NO: 1, wherein each alteration is independently a substitution and the polypeptide has lysozyme activity; and recovering the polypeptide.
56. A whole broth formulation or cell culture composition comprising a polypeptide of any of paragraphs 1-37.
57. A composition comprising the polypeptide of paragraphs 1-37.
58. The composition of paragraph 57, wherein the composition further comprises one or more components.
59. The composition according to paragraph 58, wherein the one or more component is an enzyme.
60. The composition according to paragraphs 57-59, wherein the composition is in a liquid and/or solid composition.
61. The composition according to paragraphs 57-60, wherein the composition is an animal feed composition.
62. An animal feed additive comprising
a) the polypeptide of paragraphs 1-37; and
b) at least one fat soluble vitamin, and/or
c) at least one water soluble vitamin, and/or
d) at least one trace mineral.
63. The animal feed additive of paragraph 62, which further comprises one or more enzymes.
64. Use of a polypeptide according to any of the paragraphs 1-37 in animal feed.
65. Use of a polypeptide according to any of the paragraphs 1-37 for breaking down the cell walls of bacteria.

### EXAMPLES

Chemicals used were commercial products of at least reagent grade.

### Example 1: Preparation of Variants, and Determination of Activity

### Preparation of Muramidase Variants

Expression of Muramidase Variants in Aspergillus oryzae

The constructs comprising the S. alcalophilus muramidase variant genes in the examples were used to construct expression vectors for Aspergillus. The Aspergillus expression vectors consist of an expression cassette based on the Aspergillus niger neutral amylase II promoter fused to the Aspergillus nidulans triose phosphate isomerase non translated leader sequence (Pna2/tpi) and the Aspergillus niger amyloglycosidase terminator (Tamg). Also present on the plasmid was the Aspergillus selective marker pyrG from Aspergillus nidulans enabling growth on miminal media for an aspergillus which is pyrG minus. The expression plasmids for muramidase variants were transformed into Aspergillus as described in Lassen et al. (2001), Applied and Environmental Microbiology, 67, 4701-4707. For each of the constructs 4-6 strains were isolated, purified and cultivated in microtiterplates. Expression was determined using SDS-PAGE analysis. The best producing strain was fermented in Shake flasks.

### Purification of S. Alcalophilus Muramidase Variants

The fermentation supernatant with the muramidase variant was filtered through a Fast PES Bottle top filter with a 0.22 μm cut-off. The resulting solution (100-150 ml) was diluted with MQ water to 500 ml and pH was adjusted to 4.5 with acetic acid.

After pretreatment the muramidase variant was purified by chromatography on S Sepharose, approximately 30 ml in a XK26 column, using as buffer A 50 mM sodium acetate pH 4.5, and as buffer B 50 mM sodium acetate+1 M NaCl pH 4.5. The fractions from the column were pooled based on the chromatogram monitoring absorption at 260 and 280 nm.

The molecular weight, as estimated from SDS-PAGE, was approximately 22 kDa and the purity was >95%. The molecular weights of the variants confirmed the expected mutation (with +/−1 Da).

### Lysozyme Activity Assay Using Fluorescein Conjugated Peptidoglycan

50 μl of lysozyme sample (diluted to 0.01 mg/ml in 0.01 Triton) was added to 50 μl of Fluorescein conjugated peptidoglycan (12.5 μg/ml) in 140 mM Na₂HPO₄, 40 mM citric acid, 0.01% Triton, pH 6 in a well in a microtiter plate. The plate was incubated at 30 °C for 30 minutes. During the incubation, the rate of the reaction was monitored at 485 nm (excitation)/528 nm (emission) for the 96-well plate at 2-minute intervals on a 15 SPECTRAMAX plate reader (Molecular Devices LLC). All variants were found to be active on Fluorescein conjugated peptidoglycan.

### Example 2: Screening of Variants for Thermostability

Single position variants and multiple position variants were tested for thermostability by nano Differential Scanning Fluorescence (nanoDSF) using filtered fermentation broths.

Nano-DSF monitors intrinsic tryptophan (Trp) fluorescence of the protein as a function of temperature at 330 and 350 nm. The temperature stability of a protein can be expressed by Tm (the temperature at which there is an equal population of folded and unfolded molecules) found at the inflection point of the fluorescence signal.

NanoDSF was performed with a nanoDSF Prometheus NT.48 instrument (NanoTemper Technologies GmbH, München, Germany). Muramidase variant samples (purified as described in Example 1, all of them in 50 mM Na-acetate, pH 4.5) were loaded into nanoDSF standard grade capillaries (NanoTemper Technologies GmbH; catalog number PR-C002) through capillary action. Three capillaries were filled for each sample. The capillaries were then placed into the instrument (up to 48 single capillaries can be loaded in a single run) and the laser intensity required for optimum signal generation was determined. The samples were run with the following experimental setting: temperature slope 2 °C/minute, start temperature 20 °C and end temperature 95 °C.

For a few combination variants the nanoDSF were performed in closed capillaries, here the end temperature can be extended to 110 °C.

TABLE 1

| | Tm (no of | IF | Difference |
|---|---|---|---|
| Mutations | Measurements) | to wt | to wt Tm |
| A103S | 68.6 (1) | 1.11 | 6.60 |
| A41Y | 67.6 (1) | 1.09 | 5.60 |
| C155W | 76.1 (3) | 1.23 | 14.10 |
| E118R | 76.0 (2) | 1.23 | 14.00 |
| F175S | 67.9 (2) | 1.10 | 5.90 |
| F37N | 76.6 (1) | 1.24 | 14.60 |
| F37Y | 67.2 (2) | 1.08 | 5.20 |
| G126N | 76.4 (1) | 1.23 | 14.40 |
| G126P | 67.2 (2) | 1.08 | 5.20 |
| G174P | 69.3 (2) | 1.12 | 7.30 |
| G188P | 70.4 (2) | 1.14 | 8.40 |
| H125P | 74.1 (1) | 1.20 | 12.10 |
| N153S | 68.4 (3) | 1.10 | 6.40 |
| Q143N | 68.7 (3) | 1.11 | 6.70 |
| R1Q | 67.7 (3) | 0.96 | −2.6 |
| R1T | 67.3 (2) | 1.09 | 5.30 |
| R202E | 67.8 (2) | 1.09 | 5.80 |
| T122P | 73.4 (1) | 1.18 | 11.40 |
| T14V | 72.4 (3) | 1.17 | 10.40 |
| V131C | 76.3 (2) | 1.23 | 14.30 |
| W10A | 68.3 (1) | 1.10 | 6.30 |
| W10D | 68.9 (3) | 1.11 | 6.90 |
| W10R | 74.6 (1) | 1.20 | 12.60 |
| T14V + N101S + R202E | 68.6 (2) | 1.10 | 6.00 |
| W10R + T13N + F37Y | 70.0 (2) | 1.12 | 7.40 |
| T13N + T14V + F37Y | 71.9 (1) | 1.15 | 9.30 |
| W10D + T13N + N101S | 71.2 (3) | 1.14 | 8.60 |
| W10D + N101S + V166P + Y176W | 75.0 (1) | 1.20 | 12.40 |
| W10D + V166P + Y176W | 74.5 (2) | 1.19 | 11.90 |
| W10A + T13N + T14V | 77.7 (3) | 1.24 | 15.10 |
| W10A + E118R + T122D + | 73.9 (3) | 1.18 | 11.30 |
| V166P + Y176W | | | |
| W10D + T14V + E118R + T122D | 73.2 (3) | 1.17 | 10.60 |
| W10D + V166P + Y176W + R202E | 79.1 (1) | 1.26 | 16.50 |
| W10R + T14V + N101S | 70.7 (3) | 1.13 | 8.10 |
| W10A + T13N + E118R + T122D | 69.4 (3) | 1.11 | 6.80 |
| W10D + N101S + N153S | 68.4 (1) | 1.09 | 5.80 |
| W10A + T14V + F37Y | 74.0 (1) | 1.18 | 11.40 |
| F37Y + V166P + Y176W + R202E | 73.7 (2) | 1.18 | 11.10 |
| W10A + E118R + T122D + R202E | 70.7 (1) | 1.13 | 8.10 |
| T13N + T14V + R202E | 74.9 (3) | 1.20 | 12.30 |
| W10A + T14V + N153S | 72.8 (3) | 1.16 | 10.20 |
| D6S + W10D + N101S + V166P | 69.8 (2) | 1.12 | 7.20 |
| D6S + G9P + W10D + N101S | 68.8 (3) | 1.10 | 6.20 |
| D6S + W10D + T36E + N101S | 68.2 (1) | 1.09 | 5.60 |
| G9P + W10D + N101S + F175N | 71.6 (3) | 1.14 | 9.00 |
| G9P + W10D + N101S + S129G | 70.1 (3) | 1.12 | 7.50 |
| W10D + T36E + F37E + N101S | 68.9 (1) | 1.10 | 6.30 |
| W10D + Q143E | 68.6 (1) | 1.10 | 6.00 |
| W10D + S40P + N101S + F175N | 70.7 (1) | 1.13 | 8.10 |
| W10D + Y20W + N101S + F175N | 72.2 (1) | 1.15 | 9.60 |
| I2V + G9P + W10D + N101S | 71.4 (3) | 1.14 | 8.80 |
| W10D + Q63R + A65D + N101S | 70.6 (3) | 1.13 | 8.00 |
| W10D + N101S + H125K + W131Y | 69.3 (3) | 1.11 | 6.70 |
| T13N + Y176W | 78.8 (1) | 1.12 | 8.5 |
| W10A + T14V | 79.3 (3) | 1.13 | 9 |
| W10D + T14V | 79.4 (2) | 1.13 | 9.1 |
| W10R + Y176W | 78.0 (2) | 1.11 | 7.7 |
| W10R + V166P | 78.0 (2) | 1.11 | 7.7 |
| T13N + V166P + Y176W | 77.2 (3) | 1.13 | 8.60 |
| T13N + T14V + V166P + Y176W | 83.6 (3) | 1.22 | 15.00 |
| W10A + T13N + V166P + Y176W | 80.8 (2) | 1.18 | 12.20 |
| T14V + V166P + Y176W | 80.2 (3) | 1.17 | 11.60 |
| W10A + V166P + Y176W | 79.7 (3) | 1.16 | 11.10 |
| W10D + V166P + Y176W | 80.7 (3) | 1.18 | 12.10 |
| W10A + T13N + T14V + | 87.2 (2) | 1.27 | 18.60 |
| V166P + Y176W | | | |
| F37Y + V166P + Y176W + R202E | 79.2 (2) | 1.15 | 10.60 |
| W10A + T13N + T122D + | 80.6 (1) | 1.17 | 12.00 |
| V166P + Y176W | | | |
| W10A + V166P + Y176W + R202E | 81.5 (1) | 1.19 | 12.90 |
| W10A + F37Y + V166P + Y176W | 81.8 (1) | 1.19 | 13.20 |

### Example 3: Thermostability of Variants in the pH-Range 3.0 to 8.0

Purified variants were tested for thermostability in the pH range 3.0 to 8.0 using NanoDSF (described above). The variant samples were in 0.1 M glycine, 0.1 M acetic acid, 0.1 M Bis-Tris, adjusted to the desired pH with either 0.5 M HCl or 0.1 M NaOH. The temperature slope was 3.33 °C/minute in this experiment.

Results are shown in table 2.

TABLE 2

| | pH | | | | | |
|---|---|---|---|---|---|---|
| Construct | 3.0 | 4.5 | 5.0 | 5.5 | 7.0 | 8.0 |
| wt | 53.6 | 63.4 | 63.9 | 63.7 | 61.4 | 58.7 |
| V166P Y176W | 65.2 | 75.2 | 76.5 | 76.2 | 74.8 | 73.3 |
| T14V V166P Y176W | 71.5 | 81.9 | 83.3 | 83.6 | 83.4 | 81.8 |
| W10A V166P Y176W | 75.6 | 85.5 | 86.9 | 87.1 | 87.2 | 86.1 |
| W10A T14V V166P Y176W | 76.3 | 86.4 | 87.3 | 88.0 | 89.2 | 88.5 |
| W10A T13N T14V V166P | 77.8 | 88.2 | 89.6 | 90.3 | 91.6 | 90.7 |
| Y176W | | | | | | |
| R1Q | 78.3 | 88.2 | 89.2 | 89.5 | 90.1 | 88.9 |
| R1Q W10A T13N T14V | 82.1 | 88.9 | 89.7 | 89.8 | 86.8 | 84.0 |
| V166P Y176W | | | | | | |

The variants have increased unfolding temperatures at all pH-values tested.

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description.

### Example 4: Muramidase Activity Assay

The fluorescence assay described here measures the activity muramidase wildtypes (WTs) and variants. The muramidase activity is measured using in-house synthesized substrates consisting of fluorescein isothiocyanate (FITC) labelled peptidoglycans (PGN). Micrococcus lysodeikticus ATCC No. 4698 PGN is from Sigma (cat. No. M3770). As an alternative, the FITC-PGN substrate from the Invitrogen™ EnzChek™ Lysozyme Assay Kit (cat. No. E22013) can be used.

### Assay Principle:

FITC-PGN is labeled to such a degree that the fluorescence is quenched. Muramidase action can relieve this quenching, yielding a dramatic increase in fluorescence that is proportional to muramidase activity.

The activity of muramidase is measured as the increase in fluorescence signal for 1-10 minutes using a standard plate-reader in kinetic mode. Fluorescence from a no-enzyme control and from a known muramidase reference are measured in parallel with the enzyme sample. The initial rates of reactions for sample muramidase, reference muramidase and no-enzyme control is calculated as the highest rate of change in fluorescence over time from t=0 to t=1-10 minutes. The no-enzyme control is used to correct for background signal for both sample and control by subtracting the no-enzyme initial rate value from sample and control initial rates. Calculated initial rates are normalized with the known muramidase concentrations to give a concentration normalized activity. Finally, the sample muramidase activity is divided by reference muramidase activity to give the reported relative muramidase activity (RelAct).

### Selected Materials:

In-house FITC-labelled Micrococcus lysodeikticus ATCC No. 4698 made from from Sigma M3770 material.

### Assay Protocol:

Prepare muramidase enzyme stock sample: 1 ppm in Milli-Q®-water (incl 0.01 v % Triton®X-100).

Make three additional 10-fold dilutions of the muramidase stock to get four 1.0-0.1-0.01- and 0.001 ppm enzyme samples. Prepare FITC-PGN substrate by dissolving FITC-PGN powder to a concentration of 12 μg/mL in suitable buffer, e.g. 140 mM Na₂HPO₄, 40 mM citric acid, 0.01 v % TritonX-100, pH 6.0. Mix 50 μL of each enzyme samples with 50 μL substrate. Read fluorescence signal in Biotek Synergy H1 plate reader using excitation/emission 485/528 nm for 30 minutes with fluorescence gain set to 75.

Data is evaluated is as described above, using the data values with linear range of assay response. As reference the muramidase with SEQ ID NO: 1 is used.

Results for the Sodiomyces alcalophilus muramidase wild type (SEQ ID NO: 1) and variants are are shown in the Table 4 below:

TABLE 4

| Construct | RelAct |
|---|---|
| wt | 1.0 |
| V166P Y176W | 1.8 |
| T14V V166P Y176W | 3.8 |
| W10A V166P Y176W | 2.9 |
| W10A T14V V166P Y176W | 1.4 |
| W10A T13N T14V V166P Y176W | 1.6 |
| R1Q | 1.5 |

### Example 5: Steambox Treatment

Four samples were prepared as listed in Table 3 below:

TABLE 3

| | |
|---|---|
| Sample 1 | Lysozyme with W10A/T13N/T14V/V166P/Y176W |
| | with thin salt coating |
| Sample 2 | Wildtype with thin salt coating |
| Sample 3 | Wildtype without coating |
| Sample 4 | Wild type with thick salt and oil coating |

Four samples were subjected to a laboratory scale steaming box, where the samples were exposed to a temperature of 90 degrees centigrade (°C) and 95 °C with 95% relative humidity for a conditioning time of 90 seconds.

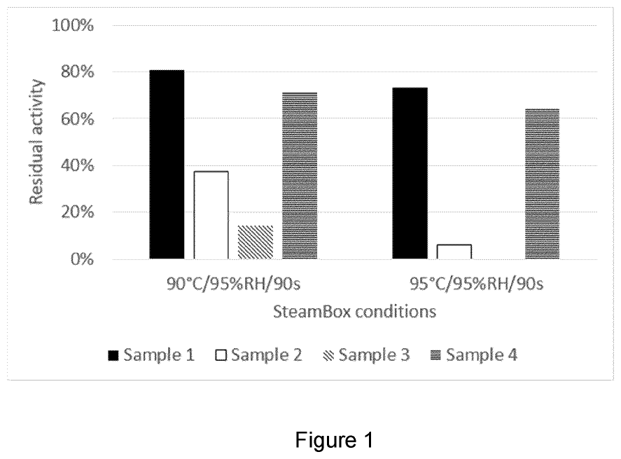

FIG. 1 shows a graph that demonstrates residual activity of the polypeptide of the invention and wildtype in steam box conditions.

Results: Sample 1 is highly stable under steambox conditions. Sample 1 has approximately 50% improvement over Sample 2 when exposed to a temperature of 90 °C, and approximately 85% improvement when exposed to a temperature of 95 °C. Sample 1 shows approximately 27% improvement compared to Sample 4 when exposed to 95 °C, whereas Sample 1 shows approximately 8% improvement compared to Sample 4 when exposed to 90 °C.

Conclusion: The substitutions of the present invention led to increased Tm and residual activity, thereby allowing for both cheaper formulations with concomitant increased activity (Sample 1 vs Sample 4) or dramatically higher activity at the same formulation (Sample 1 vs Sample 2).

1. A polypeptide having lysozyme activity comprising a substitution at one or more positions corresponding to position 1, 2, 6, 9, 10, 13, 14, 20, 36, 37, 40, 41, 63, 65, 101, 103, 118, 122, 125, 126, 129, 131, 143, 153, 155, 166, 174, 175, 176, 188 and 202 of SEQ ID NO:1, wherein the polypeptide has at least 60%, but less than 100% sequence identity to SEQ ID NO:1, or amino acids 20 to 227 of SEQ ID NO: 2.

2. The polypeptide according to claim 1, and wherein the polypeptide has improved thermostability compared to a polypeptide having SEQ ID NO: 1, or amino acids 20 to 227 of SEQ ID NO: 2.

3. The polypeptide according to claim 1, wherein the improved thermostability is measured of at least 5 °C compared to a polypeptide having SEQ ID NO:1, or amino acids 20 to 227 of SEQ ID NO: 2.

4. The polypeptide according to claim 1, wherein the polypeptide comprises one or more of the following substitutions at positions corresponding to positions 1, 10, 13, 14, 166, 176 of SEQ ID NO: 1.

5. The polypeptide according to claim 1, wherein the polypeptide comprises at least one of the following substitutions or combinations of substitutions:
R1Q, R1T, W10A, W10D, W10R, T14V, F37N, F37Y, A41Y, A103S, E118R, T122P, H125P, G126N, G126P, V131C, Q143N, N153S, C155W, G174P, F175S, G188P, R202E,
V166P+Y176W,
T14V+N101S+R202E,
W10R+T13N+F37Y,
T13N+T14V+F37Y,
W10D+T13N+N101S,
W10D+N101S+V166P+Y176W,
W10D+V166P+Y176W,
W10A+T13N+T14V,
W10A+E118R+T122D+V166P+Y176W,
W10D+T14V+E118R+T122D,
W10D+V166P+Y176W+R202E,
W10R+T14V+N101S,
W10A+T13N+E118R+T122D,
W10D+N101S+N153S,
W10A+T14V+F37Y,
F37Y+V166P+Y176W+R202E,
W10A+E118R+T122D+R202E,
T13N+T14V+R202E,
W10A+T14V+N153S,
D6S+W10D+N101S+V166P
D6S+G9P+W10D+N101S,
D6S+W10D+T36E+N101S,
G9P+W10D+N101S+F175N,
G9P+W10D+N101S+S129G,
W10D+T36E+F37E+N101S,
W10D+Q143E,
W10D+S40P+N101S+F175N,
W10D+Y20W+N101S+F175N,
I2V+G9P+W10D+N101S,
W10D+Q63R+A65D+N101S,
W10D+N101S+H125K+W131Y,
T13N+Y176W,
W10A+T14V,
W10D+T14V,
W10R+Y176W,
W10R+V166P,
T13N+V166P+Y176W,
T13N+T14V+V166P+Y176W,
W10A+T13N+V166P+Y176W,
T14V+V166P+Y176W,
W10A+V166P+Y176W,
W10A+T14V+V166P+Y176W,
W10D+V166P+Y176W,
W10A+T13N+T14V+V166P+Y176W,
F37Y+V166P+Y176W+R202E,
W10A+T13N+T122D+V166P+Y176W,
W10A+V166P+Y176W+R202E,
W10A+F37Y+V166P+Y176W, and
R1Q+W10A+T13N+T14V+V166P+Y176W
of SEQ ID NO: 1 and, wherein the polypeptide has lysozyme activity and wherein the polypeptide has at least 60%, but less than 100% sequence identity to the polypeptide of SEQ ID NO: 1 or amino acids 20 to 227 of SEQ ID NO: 2.

6. The polypeptide according to claim 1, wherein the polypeptide comprises one or more of the following substitutions at a position corresponding to positions R1Q, W10A, T13N, T14V, V166P, Y176W of SEQ ID NO: 1.

7. A polynucleotide encoding the polypeptide defined by claim 1, wherein the polynucleotide has at least 80%, but less than 100% sequence identity to SEQ ID NO: 3.

8. A nucleic acid construct comprising the polynucleotide of claim 7.

9. An expression vector comprising the polynucleotide of claim 7.

10. A host cell comprising the polynucleotide of claim 7.

11. A method of producing a polypeptide, comprising:
a. cultivating the host cell of claim 10 under conditions conducive for production of the polypeptide; and
b. recovering the polypeptide.

12. A method of obtaining a polypeptide comprising
(a) introducing a substitution at one or more positions corresponding to positions 1, 2, 6, 9, 10, 13, 14, 20, 36, 37, 40, 41, 63, 65, 101, 103, 118, 122, 125, 126, 129 131, 143, 153, 155, 166 174, 175, 176, 188 and 202 of SEQ ID NO: 1, wherein the polypeptide has lysozyme activity and has at least 60%, sequence identity to SEQ ID NO: 1 or amino acids 20 to 227 of SEQ ID NO: 2; and
(b) optionally recovering the polypeptide.

13. A composition comprising the polypeptide defined by claim 1.

14. An animal feed additive comprising
the polypeptide of claim 1; and/or
at least one fat soluble vitamin, and/or
at least one water soluble vitamin, and/or
at least one trace mineral.

15. Use of a polypeptide according to claim 1 in animal feed.


SEQUENCE LISTING (3 sequences)

SEQ ID NO: 1 - protein, 208 aa, Sodiomyces alcalophilus
RIPGFDISGWQPTTDFARAYANGDRFVYIKATEGTTFKSSAFSRQYTGATQNGFIRGAYHFAQPAASSGAAQARYFASNGGGWSKDGITLPGALDIEYNPNGATCYGLSQSAMVNWIEDFVTTYHGITSRWPVIYTTTDWWTQCTGNSNRFANRCPLWIARYASSVGTLPNGWGFYTFWQYNDKYPQGGDSNWFNGDASRLRALANGD

SEQ ID NO: 2 - protein, 227 aa, Sodiomyces alcalophilus
MKLLPSLIGLASLASLAVARIPGFDISGWQPTTDFARAYANGDRFVYIKATEGTTFKSSAFSRQYTGATQNGFIRGAYHFAQPAASSGAAQARYFASNGGGWSKDGITLPGALDIEYNPNGATCYGLSQSAMVNWIEDFVTTYHGITSRWPVIYTTTDWWTQCTGNSNRFANRCPLWIARYASSVGTLPNGWGFYTFWQYNDKYPQGGDSNWFNGDASRLRALANGD

SEQ ID NO: 3 - DNA, 624 bp, Sodiomyces alcalophilus
  source 1..624: mol_type = genomic DNA
cggatccccggctttgacatttcgggcgcccaaccgaacgtcgactttgcaagggcgtatgctaatggagatcgtttcgtctacatcaaggccaccgagggcaccacattcaagagctccgcattcagccgccagtacaccggcgcaacacaaaacggcttcatccgcggcgcctaccacttcgcccagcccgccgcgtcctcgggcgccgcgcaggcgagatacttcgccagcaacggcggcggctggtccaaggacggcatcaccctgcccggggcgctggacatcgagtacaaccccaacggcgccacctgctacggcctctcgcaatcggccatggtgaactggatcgaggactttgtcaccacctaccacggcatcacctcccgctggcccgtcatctacaccaccaccgactggtggacccagtgcaccggcaactccaaccgcttcgcgaaccgctgcccgctgtggatcgcccgctacgccagctcccctggcactctgcccaatggctggggcttttggaccttctggcagtacaacgacaagtatcctcagggcggtgattcgaactggttcaacggcgatgcgtcgcgtctcagggctctcgctaacggagac